(19) United States
(12) Patent Application Publication
(10) Pub. No.: US 2025/0236902 A1
(43) Pub. Date: Jul. 24, 2025

(54) BIOSENSORS AND USES THEREOF

(71) Applicant: Yissum Research Development Company of the Hebrew University of Jerusalem Ltd., Jerusalem (IL)

(72) Inventors: Shlomo YITZCHAIK, Jerusalem (IL); Mattan HUREVICH, Jerusalem (IL)

(73) Assignee: Yissum Research Development Company of the Hebrew University of Jerusalem Ltd., Jerusalem (IL)

(21) Appl. No.: 19/050,334

(22) Filed: Feb. 11, 2025

Related U.S. Application Data
(63) Continuation-in-part of application No. 18/852,769, filed on Sep. 30, 2024.
(60) Provisional application No. 63/552,448, filed on Feb. 12, 2024.

(30) Foreign Application Priority Data
Jun. 21, 2022 (IL) 294164

(51) Int. Cl. C12Q 1/40; G01N 27/02; G01N 27/327

(52) U.S. Cl. CPC C12Q 1/40; G01N 27/026; G01N 27/3275; G01N 2333/924; G01N 2400/00

(57) ABSTRACT

The technology disclosed herein generally concerns detection of enzymes and other biological materials by use of sialylated glycan-based detection units.

### CROSS REFERENCE TO RELATED APPLICATIONS

This application is a continuation-in-part application of U.S. patent application Ser. No. 18/852,769 filed on Sep. 30, 2024, which was a 371 of PCT/IL2023/050634 filed on Jun. 19, 2023, which claims benefit of priority from Israeli U.S. Pat. No. 294,164 filed on Jun. 21, 2022, and this application claims the benefit of priority from U.S. Provisional Patent Application No. 63/552,448 filed on Feb. 12, 2024, the contents of each of which are incorporated herein by reference in their entirety.

### TECHNOLOGICAL FIELD

The invention generally contemplates a biosensor and uses thereof.

### BACKGROUND OF THE INVENTION

Sialic acid (SA) is an important and unique monosaccharide that decorates N-glycans, O-glycans, and gangliosides on the cell membrane (generally referred to as sialosides). SA is important for cell recognition, intercellular communication, and immune system regulation. SA is also a common target for infection recognition. Neuraminidases (NAs) are enzymes that remove SA from sialosides, therefore regulate SA expression. Viral pathogens use NAs or similar proteins as part of the infection process. Viral pathogens can differentiate between cells based on the type of SA and the regiospecificity of its connection to the glycoconjugate.

There are several common approaches for evaluating NAs activity. The first approach is labeled-based. In this case, the substrate can be either fluorescently or metabolically labeled. The labeled substrate undergoes enzymatic reaction or binds the enzyme in a manner that produces a detectable signal such as fluorescence. However, this method requires large quantities of a labeled substrate and hence is limited in the use of hardly accessible sialosides. This is because their synthesis or purification from natural sources is not trivial. The second approach requires an inhibitor that binds the catalytic site and enables structural analyses. In this approach, the binding properties of the enzyme can be studied in a glycan array, for instance, which enables fingerprint patterning of the enzyme. The third approach is use of a sensory interface with a label-free technique. In this case, the substrate is attached to an interface, which produces a detectable signal upon enzyme binding or reaction such as electrochemical signals.

Electrochemical impedance spectroscopy (EIS) is a label-free electrochemical technique for evaluating interactions and biosensing. EIS relies on changes to the interfacial properties, which affect the diffusion through the layer when external RedOx active species is used. EIS is a sensitive technique that requires small amounts of material to produce a detectable signal in the sensory layer. The high sensitivity of EIS can be used for evaluating enzymatic reactions on an interface or protein binding to a substrate in the interface. This produces detectable signals with small amounts of substrate. Additionally, it was shown that enzymes can be detected by impedimetric measurements utilizing affinity to enzyme substrate or allosteric inhibitor and not just by catalytic activity. The ability to study binding and catalysis of enzymes by EIS relies on the chemistry of the interface.

A platform based on bi-antennary N-glycan was developed that enables impedimetric biosensing of sialylation and de-sialylation processes. However, that platform required time-consuming multistep modification on the oxide layer of glassy carbon electrode (GCE).

### GENERAL DESCRIPTION

Sialic acid (SA) recognition and hydrolysis are essential features of cellular function and pathogen infectivity. Neuraminidases (NAs) are viral enzymes that detach sialic acid, thereby causing viral infections. Therefore, their inhibition is a prime target for viral infection treatment, whereby the connectivity and type of sialic acid influence the recognition and hydrolysis activity towards the many different neuraminidases. This makes the effort of finding specific viral inhibitors extremely difficult. The common strategies to evaluate neuraminidase activity, recognition and inhibition rely on extensive labeling and require large amounts of sialylated glycans.

The inventors of the technology disclosed herein have developed a novel methodology for distinguishing between different neuraminidases and for evaluating their activity, recognition and inhibition. The platform proposed herein may be utilized in a greater array of analyses for being diverse and highly sensitive. The platform involves synthetic sialylated glycans that differ in the sialic acid origin and connectivity and may thus be used in label-free electrochemical impedance spectroscopy methods to differentiate between different analytes that interact with the sialic acid or the sugar backbone. Such analytes may be neuraminidases. The synthetic sialylated glycans, referred to herein as “sialosides”, can serve as tools for detecting presence and amount as well as evaluating inhibition of neuraminidases binding and enzymatic activity.

The inventors of the technology disclosed herein have synthesized several sialylated glycans, e.g., saccharides such as trisaccharide, having an active functionality, such as an amine, at the terminus on the reducing end and a variability in the sialic acid type and regiochemistry. These sialylated glycans were attached to electrode substrate of different substrate materials, e.g., a substrate of a glassy carbon electrode (GCE) or gold substrate. Differentiation of neuraminidases (NA) could be achieved based on different sialoside types (Neu5 Ac vs. Neu5GC) and regiochemistry (2-3 vs. 2-6 linkages). In other words, the platform technology of the invention makes it possible to distinguish between NAs based on the type of sialic acid and its connectivity to the core glycan. As used herein, the regiochemistry broadly refers to the sialosides' structure and not to the NAs. The different NA enzymes, originating from different viral lines and bacterial types, may bind to the sialosides with specific regioselectivity.

The modifications were characterized by EIS, contact potential difference (CPD), contact angle (CA), variable angle ellipsometry (VASE), and X-ray photoelectron spectroscopy (XPS). The modified GCEs and AuEs were exposed to two types of NAs to determine preferential response. Surface characterizations were used to elucidate if a signal received arises from enzymatic activity or binding after exposure to the enzyme. The impedimetric response was further elucidated by applying different concentrations of the NA enzyme in each system. Additionally, the effect of a NA inhibitor on the generated impedimetric signal for exposure to NA was examined.

In most general terms, the invention provides an electrode material having a sialoside functionality, namely an electrode material that is a sialylated glycan (or a glycosylated sialic acid group). The term “electrode material” broadly encompasses a material used to form an electrode for use in detection systems and devices. The electrode material may be provided as a standalone material or associated with an electrode substrate material as known in the art. The electrode substrate may be of any shape and form and is typically conductive. Non-limiting examples of electrode substrate materials include gold substrates and GCE substrates.

The electrode material is a sialoside having a glycan moiety which may be a mono-, di-, tri-, oligo- or a polysaccharide that is associated to a functionality derived from sialic acid (or which is sialic acid). Each of the glycans is given the meaning acceptable in the art. As used herein, the term “monosaccharide” refers to a simple form of a sugar that consists of a single saccharide unit which cannot be further decomposed to smaller saccharide building blocks or moieties, while the “disaccharide” is the smallest repeating backbone unit consisting of two sugar residues. The “trisaccharide” encompasses any sugar formed when three monosaccharides are joined by glycosidic linkages. The terms “oligosaccharides” and “polysaccharides” refer to saccharide polymers containing three to nine sugar residues or 10 or more sugar residues, respectively.

In some embodiments, the glycan is a sugar comprising or consisting a saccharide selected from galactose (Gal), glucose (Glc), and fructose. In some embodiments, the glycan comprises Gal and/or Glc.

In some cases, the glycan is a disaccharide or a trisaccharide, or a higher saccharide such as an oligo- or a polysaccharide comprising one or more Gal and/or Glc units. In some embodiments, the sialoside comprises a glycan having as a core structure the sequence β-D-Gal-(1-4)-β-D-GlcNAc-(1-4))-1-X, wherein X is as defined herein.

The electrode material is tailored for association on a surface region of an electrode capable of biosensing. The electrode material is thus structured to provide selective sensing of an interaction between an agent and the electrode material, wherein the interaction may be detectable by electrochemical impedance spectroscopy (EIS).

Thus, in a first of its aspects, the invention provides an electrode material comprising or consisting or being a sialoside (or a sialylated glycan), wherein the sialoside comprises (or is structured of) a glycan selected amongst monosaccharides, disaccharides, oligosaccharides, oligosaccharides and polysaccharides, as defined herein, and a sialic acid moiety.

In some embodiments, the glycan is a disaccharide, optionally a disaccharide comprising or consisting a core sequence β-D-Gal-(1-4)-β-D-GlcNAc-(1-4))-1-X, wherein Gal is galactose, Glc is glucose and X may be a C2-C10alkyl, C2-C10alkenyl, C2-C10alkynyl, a C6-C10arylene, or a C2-C10alkyl-C6-C10aryl, each being substituted by an atom or a group of atoms, being one or more of halogens (I, Br, Cl or F), hydroxy (OH), thiol (SH), disulfide (—S—S—), cyano (CN), nitro (NO2), azide (N3), amine (NH2, or a primary, secondary or tertiary amine), carboxy (COO), phosphate groups and others.

In some embodiments, in the core sequence, X is an alkyl substituted by an amine group, wherein the alkyl has between 2 and 10 carbon atoms, i.e., C2-C10alkyl-NH2.

In some embodiments, the glycan is a disaccharide comprising or consisting a core sequence β-D-Gal-(1-4)-β-D-GlcNAc-(1-4))-1-X-amine, wherein X is C2-C10alkyl. In some embodiments, the glycan is a disaccharide comprising or consisting a core sequence selected from β-D-Gal-(1-4)-β-D-GlcNAc-(1-4))-1-ethylamine, β-D-Gal-(1-4)-β-D-GlcNAc-(1-4))-1-propylamine, β-D-Gal-(1-4)-β-D-GlcNAc-(1-4))-1-butylamine, β-D-Gal-(1-4)-β-D-GlcNAc-(1-4))-1-pentylamine, β-D-Gal-(1-4)-β-D-GlcNAc-(1-4))-1-hexylamine, β-D-Gal-(1-4)-β-D-GlcNAc-(1-4))-1-heptylamine, β-D-Gal-(1-4)-β-D-GlcNAc-(1-4))-1-octylamine and others.

In some embodiments, the glycan is a disaccharide comprising or consisting a core sequence being β-D-Gal-(1-4)-β-D-GlcNAc-(1-4))-1-pentylamine or β-D-Gal-(1-4)-β-D-GlcNAc-(1-4))-1-hexylamine.

In some embodiments, X may be a C2-C10alkyl, C2-C10alkenyl, C2-C10alkynyl, a C6-C10arylene, or a C2-C10alkyl-C6-C10aryl, each being substituted by an atom or a group of atoms capable of associating to a surface region of the substrate, e.g., an electrode substrate. In some embodiments, the surface associating atom or group of atoms may be hydroxy (OH), thiol (SH), disulfide (—S—S—), azide (N3), amine (NH2, or a primary, secondary or tertiary amine), carboxy (COO), phosphate groups and others.

In some embodiments, sialosides of the invention may be regarded sialylated trisaccharides, for containing three saccharide units, one of which derived from sialic acid.

Also provided is an electrode for use in electrochemical impedance spectroscopy (EIS), the electrode having a conductive surface associated to a sialoside (or a sialylated glycan) as disclosed herein.

In some embodiments, the electrode is a gold or a GCE electrode or an electrode useful in electrochemical impedance spectroscopy (EIS), whereby a surface region of the electrode is associated with a sialoside. In some embodiments, the sialoside has a glycan moiety which may be a mono-, di-, tri-, oligo- or a polysaccharide that is associated to a functionality derived from sialic acid (or which is sialic acid).

Thus, the invention further provides an electrode comprising a gold or a GCE substrate, said substrate being associated with a sialoside, as defined herein.

In some embodiments, the sialoside comprises a glycan comprising or consisting a core sequence β-D-Gal-(1-4)-β-D-GlcNAc-(1-4))-1-X-amine, wherein X is as defined herein. In some embodiments, X is a C2-C10alkyl, substituted as disclosed herein, e.g., by an amine or a surface binding functionality.

In some embodiments, the sialoside comprises a glycan selected from β-D-Gal-(1-4)-β-D-GlcNAc-(1-4))-1-ethylamine, β-D-Gal-(1-4)-β-D-GlcNAc-(1-4))-1-propylamine, β-D-Gal-(1-4)-β-D-GlcNAc-(1-4))-1-butylamine, β-D-Gal-(1-4)-β-D-GlcNAc-(1-4))-1-pentylamine, β-D-Gal-(1-4)-β-D-GlcNAc-(1-4))-1-hexylamine, β-D-Gal-(1-4)-β-D-GlcNAc-(1-4))-1-heptylamine, β-D-Gal-(1-4)-β-D-GlcNAc-(1-4))-1-octylamine and others.

In some embodiments, the sialoside comprises the core sequence β-D-Gal-(1-4)-β-D-GlcNAc-(1-4))-1-pentylamine or β-D-Gal-(1-4)-β-D-GlcNAc-(1-4))-1-hexylamine.

As may be understood, the sialoside provided on the electrode surface acts as a barrier to charge transport from the conductive electrode substrate to the solution. In some embodiments, the electrode is one suitable for biosensing.

In some embodiments, the electrode material of the invention, as used, for example, in electrodes of the invention, is a sialoside, namely a sialylated glycan, such as a sialylated trisaccharide, wherein the group derived from sialic acid that is associated to the glycan, e.g., a disaccharide, via a linker moiety having an amine at the terminus on a reducing end and variability in the sialic acid moiety. The sialoside may comprise any human sialic acids or any monkey type sialic acids. The sialic acids may be selected from N-acetylneuraminic acid (Neu5Ac) and N-glycolylneuraminic acid (Neu5Gc).

In some embodiments, the sialic acid has an acetyl at position 5, e.g., Neu5Ac, with connectivity 2,6 (H6) and 2,3 (H3) (being human sialic acids). In some embodiments, the sialic acid has a hydroxy acetyl at position 5, e.g., Neu5Gc, with connectivity 2,6 (M6) and 2,3 (M3) (being monkey sialic acids). Other types of sialic acids may also be used.

Thus, in some embodiments, the electrode material is one or more sialosides having the Neu5Ac and/or Neu5Gc groups. In some embodiments, the electrode material is or comprises a sialoside herein designated H6, H3, M6, and M3, respectively having the structures:

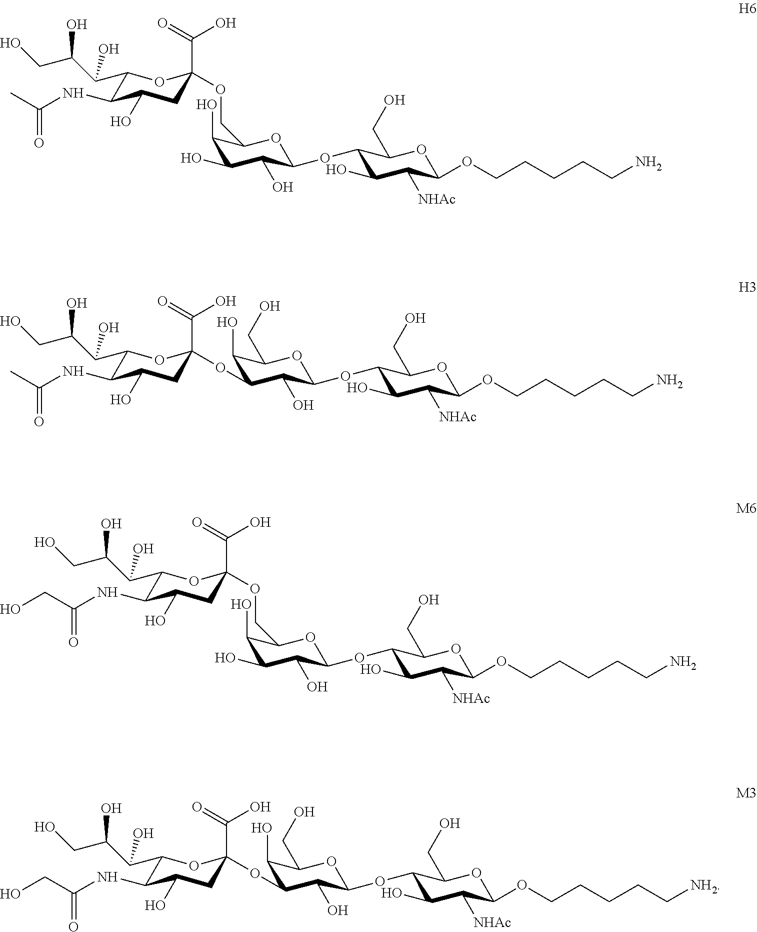

In some embodiments, the electrode of the invention is an electrode having a biosensitive surface region associated with an electrode material selected amongst sialosides herein designated H6, H3, M6 and M3. The biosensitive surface region may be any surface region of the electrode, or the complete surface of the electrode. In some cases, the electrode is suitable for EIS.

A biosensitive surface may be achievable by any chemical association protocols known in the art. Where the electrode, for example, is a gold electrode, association of the electrode material to the gold surface may be achievable by lipoic acid chemisorption and subsequent amidation with the sialylated glycan, e.g., H3, H6, M3, or M6. In another example, where the electrode is a glassy carbon electrode (GCE), electrochemical grafting of the sialylated glycan may be used.

Without wishing to be bound by theory, in order to achieve a robust association of the sialoside molecules onto a surface region of the electrode, the association typically involves chemical adsorption (or chemisorption) via surface binding groups which may be part of the sialoside structure or may be associated therewith before or during the deposition process. The surface binding groups may be amines, carboxylic acids, thiols, disulfides or generally S-containing functionalities, phosphates and generally P-containing functionalities, and others, as disclosed herein and as known in the art. Surface association may form a monolayer of the sialosides on a surface region or the complete surface of the substrate. The monolayer may be homogenous in e.g., composition and height, or may be heterogeneous, namely comprising sialosides of different compositions. In some cases, the monolayer may be formed of a mixture of different sialoside populations or types, wherein each population or type differs in composition from the other. The difference may be in the sialic acid derived moiety, the glycan, the terminal chain, and/or the surface binding group. For example, where the sialoside is a trisaccharide having the core structure β-D-Gal-(1-4)-β-D-GlcNAc-(1-4))-1-X, wherein X is as defined herein, the monolayer may comprise sialosides having the aforementioned core structure, wherein X is an alkylamine of various lengths. In other words, a monolayer may be comprised of sialosides of different compositions/lengths, wherein for each sialoside type the X is differently selected from C2-C10alkylamine.

In some embodiments, the electrode is surface modified with a monolayer of at least one sialoside population.

In some embodiments, the electrode is a gold electrode or a GCE electrode associated with a monolayer of at least one sialoside population.

Thus, further provided is a modified electrode, wherein the electrode surface is modified by a sialylated glycan, wherein the glycan is selected from monosaccharides, disaccharides and trisaccharides, associated to a sialic acid moiety being Neu5Ac or Neu5Gc. In some embodiments, the sialylated glycan is one or more of herein designated H3, H6, M3, or M6.

In some embodiments, the electrode is an array of electrodes comprising electrodes of the same type and/or electrodes modified in the same way, e.g., modified with the same sialylated glycan material. In some embodiments, an array of electrodes is provided which comprises two or more different types of electrodes.

In some embodiments, the array comprises gold-based electrodes. In some embodiments, the array comprises a GCE-based electrode. In some embodiments, the array comprises a gold-based electrode and a GCE-based electrode.

The invention further provides a detection device, e.g., an electrochemical impedance detection device, comprising an electrode according to the invention. The device may be in a form of a biosensor device which employs an electrode having a sialylated glycan surface in combination with impedance measuring elements integrated into the device. The sialylated glycan of the disclosure may be incorporated onto the surface of the electrode and a biological sample may then be flown or brought into contact with the surface of the electrode. A change in the detected impedance generally indicates agent binding or agent interaction to said sialylated glycan surface.

The detection device of the invention, being in some configurations a device for measuring a change in an electrochemical impedance of a surface, is structured with an electrode of the invention to enable analysis of interfacial properties related to biorecognition events that occur on the surface of the electrode in view of an interaction of the sialylated electrode surface with an agent or analyte, e.g., which presence and/or concentration is to be determined. The interaction which may be biocatalytic (resulting in dissociation of a sugar unit from the sialylated electrode surface) or biorecognition (resulting in an association between the sialylated surface and the agent) may be used to derive information as to the occurrence of the interaction, the type of interaction, the agent interacting with the surface, the degree and rate of interaction, the concentration of the agent present, presence of different agents, and others. Thus, the device of the invention may be exploited in a variety of fields, for achieving an effective quantitative and qualitative detection of agents such as pathogens, DNA, cancer-associated biomarkers, sugar-interacting agents, enzymes, antibodies, aptamers, cells and others.

In some embodiments, devices of the invention may be used for the detection of enzymes such as NAs in a sample. In some embodiments, the NA is a viral NA.

A difference in an electrical signal as compared to a base signal measured prior to a potential interaction between an agent and the modified electrode surface may occur due to kinetic binding of the agent to the sialylated surface or due to a change in the composition of the sialylated surface due to dissociation of the sugar backbone. As a result, electron transfer/charge transfer resistance is produced, representing the amount or concentration of bound agents or the degree of dissociation. Thus, direct and rapid determination of both biomolecular recognition actions as well as biocatalytic events is possible.

Devices of the invention may be provided as bench-top detection devices or as lab-on-a-chip devices.

Where a device of the invention comprises two or more sialylated electrodes or an array of such electrodes, both high sensitivity and high selectivity may be achievable as both biocatalytic and biorecognition events may be detected and reported. Various types of NAs can be differentiated by preferential activity with sialylated substrates, as exemplified herein. As further demonstrated, a device of the invention may be used to electrochemically evaluate enzyme preference to the sialylated surface and distinguish between different NAs. As NAs are enzymes that detach sialic acid, their inhibition is a prime target for viral infection treatment, whereby the connectivity and type of sialic acid influence the recognition and hydrolysis activity towards the NAs. Thus, use of a sialylated surface according to embodiments of the invention may be used to screen for inhibitors of NAs.

In fact, methods utilizing sialylated electrodes and devices of the invention may be used for determining presence of agents not only in the biomedical arena. In most general terms, any interaction between a sugar, represented by the sialylated glycan and an analyte may be detected.

A paradigm in array biosensing suggests that for difficult-to-discriminate analytes (e.g., analytes that are very similar and are not easy to discriminate between), it is highly beneficial to increase the number of electrode substrate types for the same set of recognition elements thus multiplying the data set without changing the number of recognition elements. Specific binding and catalysis preference towards different sialosides does exist in terms of affinity and hydrolysis kinetics. The ability to characterize each analyte such as each neuraminidase can therefore be enabled by profiling their electrochemical response towards a set of sialoside-based electrodes. Plotting the various responses on a radar plot where all the analytes respond to all detecting surfaces albeit, with different intensities can give more biodata for enabling analyte selectivity. The radar plot analysis demonstrated herein takes advantage of the different electrical signal intensities (electrochemical or chemoresistive) to provide a powerful identification tool. Radar plots presentation of multiparametric data-set provides a unique fingerprint for each analyte which allows profiling by a strait forward shape analysis.

The developed strategy allows discrimination between NAs and the evaluation of inhibitor efficacy. This strategy might be useful both for determining the infection source and for defining the anti-viral treatment against different viral strains. Standard analytical techniques rely only on sialoside structure to profile NAs.

The unique NA affinity to the surface and the sialoside layer provides a glycan-mediated protein-electrode interaction, which paves the way for identifying each NA even by using a limited set of sialosides. As exemplified, to profile NAs enzymes from different strains, eight sialoside-modified electrodes were exposed to H1N1, H3N2, and H5N1 NAs. Considering the complexity of glycan synthesis, providing means to enhance the amount of data without increasing the synthetic load is extremely important. The type and level of electrochemical impedance response is both enzyme and substrate-dependent.

As disclosed herein, protein electrochemical biosensing relies on the interactions of the binding site with a recognition moiety and is also affected by peripheral interaction surrounding the active sites on the protein and the electrode. Those peripheral interactions are determined by the surface charge, density, and other weak interactions around the primary binding site. Designing a sensing system that takes into account both a recognition moiety and surface properties, as disclosed herein, enabled the inventors to achieve a sensing selectivity that could not be obtained by focusing only on the sensing moiety. Since viral or bacterial NAs interact with many different sialosides preferentially, they cannot be accurately analyzed by a single moiety biosensing strategy. Using biosensors array and principal component analyses proved to be useful in discriminating between undistinguishable analytes, and employing the sensitive EIS methodology for the detection of NAs using a sialoside array grafted on variable surfaces enabled selective determination of NA types and set the ground for a NA inhibitor efficacy evaluation strategy.

Thus, in most general terms, the invention thus further concerns a method and a system for impedimetric detection and identification of various types of NAs by collective evaluation of binding and catalytic activity of sialoside array on at least two types of interfaces. The technology allows to evaluate a content of a sample comprising one or a plurality of NAs to determine presence of any one NA through a combination of sialoside library and electrode surface features. This also enables discriminating between bacterial and viral NAs and as well as between different viral strains or bacterial strains and also to assess inhibitor efficacy. Examples of different impedimetric fingerprints generated for a plurality of different sialosides and different substrates are shown in FIG. 22.

In another aspect the invention concerns a sensor array for discriminating between NA of different origins (e.g., different microorganisms, so as to differentiate between viral and bacterial NAs, and/or between NAs of different viral strains, and/or between NAs of bacterial strains) using electrochemical impedance spectroscopy (EIS), the array comprising a plurality of different glycated sensors (or sensing regions), each different sensor (or sensing region) being distinguishable in substrate or surface material and glycated material.

The sensor array may be an electrode or arranged as a plurality of electrodes, each defining a separate sensor or having a plurality of sensing regions, as further disclosed herein.

As used herein, the sensors or sensing regions are distinguishable in substrate material (or the material of the surface, in case the substrate and the surface thereof are not of the same material), and glycated material (namely the sialoside or glycan material forming the film on the surface). This means that the sensor array may comprise different sensors or sensing regions that differ in:
1. Variable surfaces (electrode material) with films of the same sialoside or glycan;
2. Same surfaces with different sialosides or glycans; and/or
3. Variable surfaces and different sialosides or glycans.

The invention further provides a sensor for determining presence of a NA of a particular origin, the sensor comprising a plurality of different glycated sensors (or sensing regions) each configured to detect an interaction with a different NA, wherein the plurality of different glycated sensors (or sensing regions) being distinguishable in substrate (surface) material and glycated material.

As disclosed herein, devices and methods of the invention permit facile differentiation between different NAs using electrochemical impedance spectroscopy (EIS). At the heart of the invention is a sensor array or a multiplex sensor that can receive a sample containing one or more NAs, typically of unknown origins or unknown nature, and monitor a change in an impedance signal indicative of the presence of a specific or a plurality of NAs. The device may comprise a plurality of different sensors (each differing in substrate and/or glycated material or film), or may be arranged to define a plurality of sensing regions on a common substrate, each region on substrate having a different surface material and/or glycated material or film. The NAs to be identified may be of viral or bacterial origin or may be of a specific strain of viral NAs or specific strains of bacterial strains. In other words, the technology enables discriminating between or determining whether a measured change in impedance is due to a viral NA or a bacterial NA (namely an origin of the NA), or between a specific strain of a viral or a bacterial NA (namely the nature of the NA).

Interaction between a NA and one or more of the sensors or sensing regions sensor regions in a device of the invention generates a change in the measurable impedimetric signal that may be attributed to a single NA. The change in the impedimetric signal which may be due to interaction with the glycated sensor may be an increase or a decrease in the impedimetric signal measured for the glycated sensor. Such a change may be unique to a given NA and thus can distinguish between different NAs. When a tested sample comprises two or more NAs, the impedimetric signal may be a linear combination of the different NAs. A change in the impedimetric signal may further enable, as further disclosed herein, to differentiate between pathogenic NAs, and thus differentiate between diseases, disease states, can provide insight as to the state of a disease or progression thereof, can identify onset of a disease at an early stage before symptoms develop and can assist in determining success of a therapeutic treatment (prophylaxis or treatment of existing symptoms).

The ability to uniquely and unequivocally differentiate between different NAs resides in a combination of sialoside library and electrode surface features, defining in combination a large variety of glycated sensors or sensing surfaces or regions that ensure or improve the probability of a measurable interaction between an NA and at least one of the glycated sensing regions making up the glycated sensor. The library comprises for a given sensor array of the invention a collection of impedimetric signals, each corresponding to a different NA.

The invention thus further provides a library of impedimetric signals, the library comprising a collection or a listing of impedance response patterns, each corresponding to a specific NA, wherein the impedance response patterns are characterized by one or more impedance values measured at one or more frequencies or over one or more time intervals, and wherein the library is stored in a digital or electronic format, allowing retrieval and comparison of the impedance response patterns with impedance signals obtained from a sensor array according to the invention.

The invention further provides a system for detecting and determining presence (or nature) of at least one NA, the system comprising a plurality (at least two, or two or more) of sensors (or sensing regions) within a sensor array of the invention, each sensor (or sensing regions) having a predefined glycated surface configured to detect an impedance signal indicative of a respective NA; a library of impedimetric signals, wherein the library comprises a collection of predefined impedance response patterns corresponding to different NAs; a signal processing unit in communication with the sensor array, the processing unit configured to compare the impedance signals from the sensors to the impedance signals in the library to identify the detected NA, wherein the sensor array is adapted to provide a real-time impedance response that is matched to one or more impedance signals from the library, thereby enabling identification of the NA based on the impedance response.

Further provided is a non-transitory computer-readable storage medium on which is stored a library according to the invention. Typically, the non-transitory computer-readable storage medium is a tangible, physical device that can store the data. The non-transitory storage medium containing the library may be a hard drive, a flash drive, an optical disc (e.g., CDs, DVDs), a memory card, or a solid-state drive (SSDs), just to name a few.

Further provided is a method of using a non-transitory computer-readable storage medium, the method comprising storing on the non-transitory computer-readable storage medium a library comprising reference data according to the invention; receiving tested data relating to potentially presence of one or more NA; comparing the tested data with the reference data stored in the library; optionally generating a comparison result based on the comparison between the tested data and the reference data; and outputting the comparison result to an external device for display or further manipulation.

In some cases, the non-transitory medium may comprise a set of predefined rules, models, or datasets such as trained models, thresholds, or algorithms that can be used by a machine learning model to interpret the tested data.

As noted herein, a sensor of the invention comprises a plurality of different glycated sensors or sensing regions. The different sensors or regions may be part of the same sensing surface or may be a collection of different sensors arrayed together to provide the sensor device. Irrespective of the specific construction of the device, the device should comprise a plurality of different sensing regions that are differentiated in the glycated nature thereof. Reference to different “glycated sensor regions” or different glycated regions means different sensors or sensing regions that are distinguishable in both the material of the substrate onto which the NA recognition material (i.e., sialosides) are attached and the recognition materials themselves.

The different recognition materials are synthetic sialylated glycans that differ in the sialic acid origin and connectivity, as disclosed herein.

In some embodiments, the glycan is a sugar comprising or consisting a saccharide selected from galactose (Gal), glucose (Glc), and fructose. In some embodiments, the glycan comprises Gal and/or Glc.

In some cases, the glycan is a disaccharide or a trisaccharide, or a higher saccharide such as an oligo- or a polysaccharide comprising one or more Gal and/or Glc units. In some embodiments, the sialoside comprises a glycan having as a core structure the sequence β-D-Gal-(1-4)-β-D-GlcNAc-(1-4))-1-X, wherein X is as defined herein.

Thus, in some embodiments, the glycated sensor is a sensor or an electrode having a film of a sialoside (or a sialylated glycan), wherein the sialoside comprises (or is structured of) a glycan selected amongst monosaccharides, disaccharides, oligosaccharides, oligosaccharides and polysaccharides, as defined herein, and a sialic acid moiety.

In some embodiments, the glycan is a disaccharide, optionally a disaccharide comprising or consisting a core sequence β-D-Gal-(1-4)-β-D-GlcNAc-(1-4))-1-X, wherein Gal is galactose, Glc is glucose and X may be a C2-C10alkyl, C2-C10alkenyl, C2-C10alkynyl, a C6-C10arylene, or a C2-C10alkyl-C6-C10aryl, each being substituted by an atom or a group of atoms, being one or more of halogens (I, Br, Cl or F), hydroxy (OH), thiol (SH), disulfide (—S—S—), cyano (CN), nitro (NO2), azide (N3), amine (NH2, or a primary, secondary or tertiary amine), carboxy (COO), phosphate groups and others.

In some embodiments, X is an alkyl substituted by an amine group, wherein the alkyl has between 2 and 10 carbon atoms, i.e., C₂-C10alkyl-NH2.

In some embodiments, the glycan is a disaccharide comprising or consisting a core sequence β-D-Gal-(1-4)-β-D-GlcNAc-(1-4))-1-X-amine, wherein X is C2-C10alkyl. In some embodiments, the glycan is a disaccharide comprising or consisting a core sequence selected from β-D-Gal-(1-4)-β-D-GlcNAc-(1-4))-1-ethylamine, β-D-Gal-(1-4)-β-D-GlcNAc-(1-4))-1-propylamine, β-D-Gal-(1-4)-β-D-GlcNAc-(1-4))-1-butylamine, β-D-Gal-(1-4)-β-D-GlcNAc-(1-4))-1-pentylamine, β-D-Gal-(1-4)-β-D-GlcNAc-(1-4))-1-hexylamine, β-D-Gal-(1-4)-β-D-GlcNAc-(1-4))-1-heptylamine, β-D-Gal-(1-4)-β-D-GlcNAc-(1-4))-1-octylamine and others.

In some embodiments, the glycan is a disaccharide comprising or consisting a core sequence being β-D-Gal-(1-4)-β-D-GlcNAc-(1-4))-1-pentylamine or β-D-Gal-(1-4)-β-D-GlcNAc-(1-4))-1-hexylamine.

In some embodiments, X may be a C2-C10alkyl, C2-C10alkenyl, C2-C10alkynyl, a C6-C10arylene, or a C2-C10alkyl-C6-C10aryl, each being optionally substituted by an atom or a group of atoms capable of associating to a surface region of the substrate, e.g., an electrode substrate. In some embodiments, the surface associating atom or group of atoms may be hydroxy (OH), thiol (SH), disulfide (—S—S—), azide (N3), amine (NH2, or a primary, secondary or tertiary amine), carboxy (COO), phosphate groups and others.

In some embodiments, X may be a C2-C10alkyl-NH-Y, wherein Y is selected from C2-C10alkyl, C2-C10alkenyl, C2-C10alkynyl, a C6-C10arylene, or a C2-C10alkyl-C6-C10aryl, each being optionally substituted by an atom or a group of atoms capable of associating to a surface region of the substrate, e.g., an electrode substrate. In some embodiments, the surface associating atom or group of atoms may be hydroxy (OH), thiol (SH), disulfide (—S—S—), azide (N3), amine (NH2, or a primary, secondary or tertiary amine), carboxy (COO), phosphate groups and others.

In some embodiments, X may be a C2-C10alkyl-NH-Y, wherein Y is selected from C2-C10alkyl-(SH)n, wherein n designates a number of SH groups. In some embodiments, n is 1 or 2. In some embodiments, Y is —(C—O)-C2-C10alkyl-SB, wherein SB is a surface associating atom or group of atoms may be hydroxy (OH), thiol (SH), disulfide (—S—S—), azide (N3), amine (NH2, or a primary, secondary or tertiary amine), carboxy (COO), phosphate groups and others.

In some embodiments, sialosides of the invention may be regarded sialylated trisaccharides, for containing three saccharide units, one of which derived from sialic acid.

In some embodiments, the glycated sensor comprises a surface attached sialylated trisaccaride attached to a surface. The sialylated trisaccaride having a sialoside selected from β-D-Gal-(1-4)-β-D-GlcNAc-(1-4))-1-ethylamine, β-D-Gal-(1-4)-β-D-GlcNAc-(1-4))-1-propylamine, β-D-Gal-(1-4)-β-D-GlcNAc-(1-4))-1-butylamine, β-D-Gal-(1-4)-β-D-GlcNAc-(1-4))-1-pentylamine, β-D-Gal-(1-4)-β-D-GlcNAc-(1-4))-1-hexylamine, β-D-Gal-(1-4)-β-D-GlcNAc-(1-4))-1-heptylamine, β-D-Gal-(1-4)-β-D-GlcNAc-(1-4))-1-octylamine and others, wherein the amine may be further substituted by —(C—O)-C2-C10alkyl-SB, wherein SB is a surface associating atom or group of atoms may be hydroxy (OH), thiol (SH), disulfide (—S—S—), azide (N3), amine (NH2, or a primary, secondary or tertiary amine), carboxy (COO), phosphate groups and others.

In some embodiments, the sialylated glycan, such as a sialylated trisaccharide, comprises a group derived from sialic acid that is associated to the glycan, e.g., a disaccharide, via a linker moiety having an amine at the terminus on a reducing end and variability in the sialic acid moiety. The sialoside may comprise any human sialic acids or any monkey type sialic acids. The sialic acids may be selected from N-acetylneuraminic acid (Neu5Ac) and N-glycolylneuraminic acid (Neu5Gc).

In some embodiments, the sialic acid has an acetyl at position 5, e.g., Neu5Ac, with connectivity 2,6 (H6) and 2,3 (H3) (being human sialic acids). In some embodiments, the sialic acid has a hydroxy acetyl at position 5, e.g., Neu5Gc, with connectivity 2,6 (M6) and 2,3 (M3) (being monkey sialic acids). Other types of sialic acids may also be used.

While the present technology is exemplified by different sialoside recognition elements, other recognition elements may be alternatively and similarly used. Such recognition units may be glycans, peptides, proteins, oligonucleotides, small molecules, and others.

Also, in addition to or alternatively to detecting NAs, other analytes may be similarly detected. Such include proteins: including mutations, post-translationally modified proteins, and more; enzymes: cellular and extracellular; pathogens: viruses, bacteria, fungi and more; cells: including tumor cells immune cells muscle cells; and others.

The substrate surface onto which the sialosides are deposited or attached, as disclosed herein, may be any electrode material that is typically conductive and which is suitable for EIS measurements. Non-limiting examples of electrode materials include gold, glassy carbon (GC), indium-tin-oxide (ITO), chromium, graphite, silicone (including microporous and nanowires), single wall and multi wall carbon nanotubes (CNT), silver (including nanoparticles and nanorods), iron oxide (Fe₃O₄), graphene (including chitosan composites), conducting polymers (e.g., polyaniline and PEDOT) and others.

In some embodiments, the substrate (surface) material is gold or GC.

In some embodiments, the sensor array of the invention, irrespective of its configuration and structure, comprises a plurality of different sensing regions or different sensors, differing in the surface material (or electrode material) being a gold surface or a glassy carbon surface and the glycated film form thereon (namely the type of sialoside material). For example, a sensor array may comprise a plurality (two or more) of different sensing regions, wherein the sensing regions may be as follows:

| Sensing region | | |
|---|---|---|
| or sensor | Surface material | Sialoside |
| 1 | Gold | H6 |
| 2 | Gold | H3 |
| 3 | Gold | M6 |
| 4 | Gold | M3 |
| 5 | Glassy carbon | H6 |
| 6 | Glassy carbon | H3 |
| 7 | Glassy carbon | M6 |
| 8 | Glassy carbon | M3 |
| 9 | Gold | β-D-Gal-(1-4)-β-D-GlcNAc-(1-4))- |
| | | 1-ethylamine |
| 10 | Gold | β-D-Gal-(1-4)-β-D-GlcNAc-(1-4))- |
| | | 1-propylamine |
| 11 | Gold | β-D-Gal-(1-4)-β-D-GlcNAc-(1-4))- |
| | | 1-butylamine |
| 12 | Gold | β-D-Gal-(1-4)-β-D-GlcNAc-(1-4))- |
| | | 1-pentylamine |
| 13 | Gold | β-D-Gal-(1-4)-β-D-GlcNAc-(1-4))- |
| | | 1-hexylamine |
| 14 | Gold | β-D-Gal-(1-4)-β-D-GlcNAc-(1-4))- |
| | | 1-heptylamine |
| 15 | Gold | β-D-Gal-(1-4)-β-D-GlcNAc-(1-4))- |
| | | 1-octylamine |
| 16 | Glassy carbon | β-D-Gal-(1-4)-β-D-GlcNAc-(1-4))- |
| | | 1-ethylamine |
| 17 | Glassy carbon | β-D-Gal-(1-4)-β-D-GlcNAc-(1-4))- |
| | | 1-propylamine |
| 18 | Glassy carbon | β-D-Gal-(1-4)-β-D-GlcNAc-(1-4))- |
| | | 1-butylamine |
| 19 | Glassy carbon | β-D-Gal-(1-4)-β-D-GlcNAc-(1-4))- |
| | | 1-pentylamine |
| 20 | Glassy carbon | β-D-Gal-(1-4)-β-D-GlcNAc-(1-4))- |
| | | 1-hexylamine |
| 21 | Glassy carbon | β-D-Gal-(1-4)-β-D-GlcNAc-(1-4))- |
| | | 1-heptylamine |
| 22 | Glassy carbon | β-D-Gal-(1-4)-β-D-GlcNAc-(1-4))- |
| | | 1-octylamine |

In a similar way, other combinations of a substrate (surface) material and a sioloside may be used in an array of the invention.

In some embodiments, the sensor array or device comprises a plurality of different sensors or sensing regions, wherein at least two of the sensors or regions are selected from sensors or sensing regions 1-22 listed in the table above.

The number of different sensors or sensing regions may vary and is unlimited. Typically, the number of sensors or sensing regions is not one, or is two or two or more. In some embodiments, the number of sensors or sensing regions is 2, 3, 4, 5 or higher. In some embodiments, the number of sensors or sensing regions is at least 2, at least 3, at least 4, or at least 5. In some embodiments, the number of sensors or sensing regions is between 2 and 5 or between 5 and 10 or any other higher number of sensors or sensing regions.

In some embodiments, the sensor array comprises a plurality of sensors or sensing regions, some of which having gold surfaces, some having glassy carbon surfaces and optionally some having surfaces of a different material, wherein each of the surfaces being provided with a film of at least one sialoside, as defined herein.

In some cases, some of the sensors or sensing regions having gold surfaces are provided with a film of one silaoside, while other sensors or sensing regions having gold surfaces are provided with a film of a different silaoside. Independently or in combination, some of the glassy carbon surfaces may be provided with a film of one silaoside, while other sensors having glassy carbon surfaces are provided with a film of a different sialoside.

In some embodiments, the sensor array may be comprised of sensors or sensing regions of two or more different surface materials and two or more different silaoside.

The sialylated glycan of the disclosure may be incorporated onto the different sensors or sensing regions and a sample, e.g., a biological sample may then be flown or brought into contact with the surface. A change in the detected impedance generally indicates agent binding or agent interaction to said sialylated glycan surface. A detection device of the invention, being in some configurations a device for determining presence of a particular NA is based on a measurable change in an electrochemical impedance of a surface due to biorecognition events that occur on the surface of the electrode in view of an interaction of the sialylated electrode surface with an agent or analyte, e.g., NA which presence is to be determined. The interaction which may be biocatalytic (resulting in dissociation of a sugar unit from the sialylated electrode surface) or biorecognition (resulting in an association between the sialylated surface and the agent) may be used to derive information as to the NA that causes the change in the measurable signal, based on the library discussed herein.

A difference in an electrical signal as compared to a base signal measured prior to a potential interaction between an NA and the sensing surface(s) may occur due to kinetic binding of the NA to the sialylated surface or due to a change in the composition of the sialylated surface due to dissociation of the sugar backbone. As a result, electron transfer/charge transfer resistance is produced, representing the amount or concentration of bound agents or the degree of dissociation. Thus, direct and rapid determination of both biomolecular recognition actions as well as biocatalytic events is possible.

Devices of the invention may be provided as bench-top detection devices or as lab-on-a-chip devices.

In another aspect, the invention thus provides a method for determining an interaction between a sugar, namely the sialylated glycan, e.g., sialylated trisaccharide, and an agent/analyte, wherein the method comprises contacting a sialylated electrode surface, as disclosed herein, with a sample containing or suspected of containing said agent and determining a change in an impedance signal generated from a base signal obtained for a control sample. The change is likely to result from an interaction, as disclosed herein, between the sugar, i.e., the sialylated surface of the electrode and the agent.

The “agent” which detection is desired may be any such material which can chemically or physically interact with the sialylated glycan associated to a surface region of the electrode to produce a measurable resistance or a measurable change in a surface impedance. As noted herein, the agent may interact with the sialylated glycan by binding to a region of the sugar moiety or by directly or indirectly causing a structural change in the sialylated glycan, e.g., by bond scission. Depending on the sample or environment in which the agent is present, or suspected of being present, the agent may be any solid, liquid or gaseous material of an environmental origin, a biological origin, a natural origin, a synthetic origin or of an unknown origin.

Where elements of the invention (such as electrodes and devices) are used in the medical arena, biochemical or chemical agents may be detected. Such include inhibitors, chemical ablation agents, toxins, pathogens, immunomodulators, cytokines, cytotoxic agents, chemotherapeutic agents, drugs and therapeutic agents, DNA, cancer-associated biomarkers, peptides and proteins, enzymes, antibodies, aptamers, cells, glycans and glycol-conjugates and others. Where elements of the invention are used in an environmental setting, e.g., for detection of environmentally present agents, chemical such as toxins, toxic gases, free radicals, body odors and body odor volatiles, volatile organic compounds, metal ions and metalloproteins and others may be detected.

The agent may be presented to a surface of an electrode of the invention in a carrier medium, which does not by itself generate a measurable single, or which can be used as a control or background signal for determining presence of an agent carried in a sample of similar constitution. The medium may be water or containing water. Samples of unknown composition may be diluted in a buffer and tested.

As generally disclosed herein, methods of the invention comprise contacting an electrode, as defined herein, with a sample containing or suspected of containing an agent and determining a change in an impedance signal generated from a base signal obtained for a control sample. The control sample used for determining a set point, a threshold or a background signal from which a signal may be attributed to the agent may be determined, may be a carrier medium identical to that of the sample known not to contain the agent, an identical sample known not to contain the agent, or generally any agent-free sample. For example, where presence of a viral NA is a biological sample, e.g., blood sample, is desired, a blood sample known not to contain the viral NA may be used as a control.

Similarly, for determining concentration of an agent or an improvement in a medical treatment following or during treatment, e.g., antiviral treatment, a subject's blood samples may be testes on numerous occasions during a period the subject is undergoing the medical treatment to detect changes in the concentration of the NA, as determined by a change in impedance, whereby such a change may indicate a reduction in the amount of NA present in the blood of the subject. A determination of concentration may be based on predetermined studies using varying known concentrations of the agent to be detected, as known and practiced in the art.

The invention further provides a method for determining an amount or a concentration of an agent in a sample, the agent being capable of interacting with a sialylated glycan, e.g., sialylated trisaccharide, surface (an electrode surface coated or associated with the glycan), the method comprising contacting said surface with the sample, measuring a change in an impedance signal relative to an impedance signal measured for one or more control samples having known concentrations of the agent and determining concentration of the agent in the sample.

Also provided is a method of screening for a viral inhibitor, the method comprising contacting a sialylated glycan, such as a sialylated trisaccharide electrode surface, as disclosed herein, with a potential viral inhibitor in presence of a viral pathogen or a neuraminidase (NA) and determining a change in an impedance signal generated upon contact with the glycan provided on the electrode surface, wherein a change in the signal from a base signal obtained for a control sample not containing the pathogen or NA and/or not containing the potential viral inhibitor indicates inhibition or absence of inhibition of the pathogen or NA by the inhibitor. In other words, where in the presence of the potential viral inhibitor a change in the signal is not observed, the indication may be successful partial or full inhibition of the pathogen or NA. Where a change in the signal is observed, that change may support that the potential inhibitor is in fact not capable of inhibition.

The change in the impedance signal as compared to a base signal generated for a control sample provides an indication of an interaction between an agent and the sialylated glycan surface. The change on the signal may be an increase or a decrease in the impedance signal relative to a base line of the glycated surface under buffered solution (enzyme free). An increase of the impedance signal may suggest adsorption or association of the agent, e.g., an enzyme, to the sialylated glycan (via biorecognition pathways), while a decrease in the signal may suggest dissociative interaction (via biocatalytic pathways).

In some embodiments, methods and systems of the invention are tailored for biomedical uses, including screening for active drugs, determining presence or absence of biological markers in a sample, presence or absence of pathogens or toxins in a sample, etc.

In some embodiments, methods and systems of the invention may be used for screening for enzyme inhibitors. An enzyme inhibitor may be any material which reduces a rate of an enzyme catalyzed reactions by interfering with the enzyme in some way. In some embodiments, the enzyme inhibitor is an antiviral, antibiotic, anticancer drug, e.g., which screening is desired.

In some embodiments, the enzyme inhibitor is an inhibitor of NA, as defined herein.

In some embodiments, the enzyme inhibitor is an inhibitor of sialyltransferase.

The invention further provides a kit comprising a sialoside of the invention and instructions permitting association thereof onto a surface region of a substrate.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIGS. 1A-D provide structures of trisaccharides used in accordance with some embodiments of the invention: A) 2,6-Neu5Ac-Gal-GlcNAc trisaccharide (H6), B) 2,3-Neu5Ac-Gal-GlcNAc trisaccharide (H3), C) 2,6-Neu5Gc-Gal-GlcNAc trisaccharide (M6), D) 2,3-Neu5Gc-Gal-GlcNAc trisaccharide (M3).

FIGS. 2A-B provide A) Synthesis of donor 8. Reagents and conditions: (a) i) Ac₂O, pyridine, rt, 12 h; ii) Cs₂CO₃, AllylBr, DMF, 40° C., 4 h, 69% over two steps; (b) p-Thiocresol, BF₃·OEt₂, CH₂Cl₂, rt, 24 h, 78%; (c) Boc₂O, DMAP, THF, 60° C., 4 h, 85%; (d) NaOMe, AllylOH, rt, 4 h, 56%; (e) i) TFA/CH₂Cl₂ (1:1, v/v), rt, 3 h ii) NO₂C₆H₄OCOCl, NaHCO₃, H₂O/MeCN (2:1, v/v), 0° C., 4 h, 47% over two steps; (f) Ac₂O, pyridine, rt, 12 h, 84%; (g) Acetoxyacetyl chloride, DIPEA, CH₂Cl₂, 0° C. to rt, 2 h, 83%; (h) NIS, TfOH, dibutyl phosphate, CH₂Cl₂, 0° C., 6 h, 74%. B) Synthesis of M6 and M3 trisaccharides. Reagents and conditions: (i) 12, TMSOTf, CH₂Cl₂, −50° C., 2 h, 72%; (j) 19, NIS, TfOH, CH₂Cl₂, −20° C., 2 h, 21: 71%; 26: 68%; (k) Zn, THF/AcOH/Ac₂O (3:2:1, v/v), rt, 4 h, 22: 65%, 27: 70%; (1) 1,2-ethanedithiol, DBU, CH₂Cl₂, 0° C., 2 h, 23: 74%; 28: 75%; (m) i) LiOH, THF/H2O/MeOH (2:2:1, v/v), rt, 12 h; ii) Pd(OH)₂/C, H₂, H₂O/MeOH (3:1, v/v), rt, 48 h, 24: 63%; 29: 59%, over two steps. (n) i) 17, TMSOTf, CH₂Cl₂, −50° C., 2 h; ii) Ac₂O, pyridine, rt, 12 h, 49% over two steps.

FIGS. 3A-B depict A) electrografting process of GCE with i) amino terminated trisaccharide applying 5 cycles of scans from 0.6 to 1.2 V at scan rate of 0.01 V/s B) modification of AuE with trisaccharides in two steps: ii) self-assembly of LPA and iii) coupling the amino terminated trisaccharide with the LPA using COMU.

FIGS. 4A-D depict A) Nyquist plot of Impedimetric response of H3 before and after exposure to 3NACP. B) Normalized R_{CT} for the response of GCE H6 and H3 after exposure to 3 mU/mL 6NAAU or 3NACP. C) Nyquist plot of Impedimetric response of H3 before and after exposure to 3NACP. D) Normalized R_{CT} for the response of GCE H6 and H3 after exposure to 3 mU/mL 6NAAU or 3NACP. Errors are the standard deviation of 5 electrodes.

FIG. 5 provides a heat map of the response magnitude after exposure the substrates to the enzyme in different platforms. The response is in Normalized R_{CT}.

FIGS. 6A-B provide A) Normalized R_{CT} of concentration dependent response to the 3NACP with GCE-H3. B) Normalized R_{CT} of concentration dependent response to the 3NACP with AuE-H3.

FIGS. 7A-B provide A) Normalized R_{CT} of impedimetric response of GCE-H3 to 0.3 mU/mL 3NACP with different concentrations of Oseltamivir. B) 1/Normalized R_{CT} of impedimetric response of AuE-H3 to 3 mU/mL 3NACP with 1 μM Oseltamivir and without.

FIG. 8 provides a schematic summary of the sensing ability using AuE and GCE. AuE modified with the trisaccharide is responsive to the enzymatic reaction while GCE modified with the trisaccharide is responsive to enzyme binding.

FIGS. 9A-B depict A) Influenza utilizes Hemagglutinin to target sialylated glycan on cell surface and uses neuraminidase to detach from the cell by cleaving sialic acid. B) Systems used in this work to target Influenza neuraminidase.

FIG. 10 depicts modified GCE and AuE with the trisaccharides according to some embodiments of the invention.

FIGS. 11A-B shows Nyquist plot of response to H1N1 NA by A) GCE-H3 and by B) AuE-H3 where green plot is prior to exposure and red is after exposure.

FIGS. 12A-B depicts normalized R_{CT} response to H1N1 and H3N2 NA by A) GCE modified with sialosieds B) AuE modified with sialosides. The standard deviation is based on response of 5 electrodes.

FIG. 13 shows a heatmap of Log (Normalized R_{CT}) for the response of different NA with the presented sensory layers.

FIGS. 14A-E provide a Radar plot of |Log (Normalized R_{CT})| for the response of H1N1 (A), H3N2 (B), H5N1 (C), NACP (D), and NAAU (E) with the presented sensory layers.

FIG. 15 provides normalized R_{CT} response of AuE-H3 to H3N2 NA in presence and absence of Viral NA inhibitors.

FIG. 16 provides the modified GCE layers, which was formed as hydrophobic glycated layer by electrodeposition of aminyl trisaccharides, and AuE layers, which was formed as a negatively charged glycated layer by amidation of aminyl trisaccharides with LPA, that were used in this work.

FIGS. 17A-B are schematic representation of NA interaction modes: (a) NA with deep catalytic site also experiences hydrophobic interactions between protein interface and electrode (red) and (b) NA with surface exposed catalytic site does not experience additional interactions between the protein and the electrode surface.

FIGS. 18A-B provide XPS analyses of N1s for Au-H3 prior (a) and after (b) exposure to neuraminidase H3N2 with peak at 400.1 corresponding to N1s of amide of the sialoside without addition of amide corresponding to the presence of protein on the surface.

FIG. 19 provides Psi of VASE analyses of Au-LPA and Au-LPA-H3 prior and after exposure to neuraminidase H3N2 where the line is fit and the circles are raw data. Insert contains zoom to the region with the largest difference.

FIG. 20 provides Delta of VASE analyses of Au-LPA and Au-LPA-H3 prior and after exposure to neuraminidase H3N2 where the line is fit and the circles are raw data. Insert contains zoom to the region with the largest difference.

FIGS. 21A-B provides XPS analyses of NIS for GCE-H3 prior (A) and after (B) exposure to neuraminidase H3N2. Where red correlates with ammonium, blue with amide, and green with nitride. Increase of amide on GCE at peak of 400.1 corresponds to attachment of protein to the surface.

FIGS. 22A-Y provide exemplary impedimetric fingerprints for different NAs measured for different glycated surfaces according to the invention: (A) Nyquist plot of GCE-H3 with H3N2 neuraminidase before and after exposure; (B) Nyquist plot of GCE-H6 with H3N2 neuraminidase before and after exposure; (C) Nyquist plot of GCE-M3 with H3N2 neuraminidase before and after exposure; (D) Nyquist plot of GCE-M6 with H3N2 neuraminidase before and after exposure; (E) Nyquist plot of GCE-H6 with H1N1 neuraminidase before and after exposure; (F) Nyquist plot of GCE-M3 with H1N1 neuraminidase before and after exposure; (G) Nyquist plot of GCE-M6 with H1N1 neuraminidase before and after exposure; (H) Nyquist plot of GCE-H3 with H5N1 neuraminidase before and after exposure; (I) Nyquist plot of GCE-H6 with H5N1 neuraminidase before and after exposure; (J) Nyquist plot of GCE-M3 with H5N1 neuraminidase before and after exposure; (K) Nyquist plot of GCE-M6 with H5N1 neuraminidase before and after exposure; (L) Nyquist plot of AuE-H3 with H3N2 neuraminidase before and after exposure; (M) Nyquist plot of AuE-H6 with H3N2 neuraminidase before and after exposure; (N) Nyquist plot of AuE-M3 with H3N2 neuraminidase before and after exposure; (O) Nyquist plot of AuE-M6 with H3N2 neuraminidase before and after exposure; (P) Nyquist plot of AuE-H6 with H1N1 neuraminidase before and after exposure; (Q) Nyquist plot of AuE-M3 with H1N1 neuraminidase before and after exposure; (R) Nyquist plot of AuE-M6 with H1N1 neuraminidase before and after exposure; (S) Nyquist plot of AuE-H3 with H5N1 neuraminidase before and after exposure; (T) Nyquist plot of AuE-H6 with H5N1 neuraminidase before and after exposure; (U) Nyquist plot of AuE-M3 with H5N1 neuraminidase before and after exposure; (V) Nyquist plot of AuE-M6 with H5N1 neuraminidase before and after exposure; (W) Nyquist plot of AuE-H3 with H3N2 neuraminidase with presence of oseltamivir before and after exposure; (X) Nyquist plot of AuE-H3 with H3N2 neuraminidase with presence of zanamivir before and after exposure; (Y) Nyquist plot of AuE-H3 with H3N2 neuraminidase with presence of peramivir before and after exposure.

### DETAILED DESCRIPTION OF EMBODIMENTS

### Part I: Preparation of Recognition Surfaces

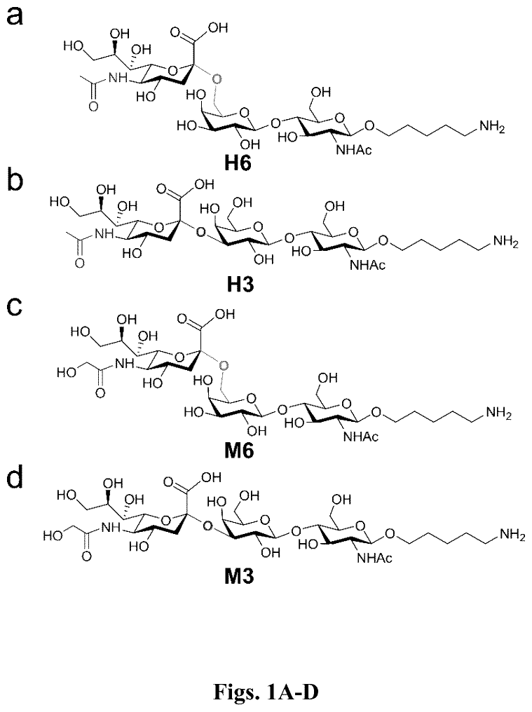

Various types of neuraminidases (NA) can be differentiated by preferential activity with sialylated substrate. Four trisaccharide substrates were synthesized via multistep processes to allow high control of the regiochemistry on a common core sialosides (FIG. 1). The four substrates containing the same core structure of 6-(β-D-Gal-(1-4)-β-D-GlcNAc-(1-4))-1-amine with two types of sialic acid and two types of connectivity. Two human sialosides are known to contain acetyl at position 5, namely Neu5Ac, with connectivity 2,6 (H6) and 2,3 (H3) and there are two monkey type sialosides with hydroxy acetyl at position 5, namely Neu5Gc with connectivity 2,6 (M6) and 2,3 (M3) (FIG. 1). The chemical synthesis of sialic acid glycans is a formidable synthetic challenge due to its instability, difficulties in α-glycosylation and low reactivity. Using a similar synthetic strategy sialic acid donor, Neu5Ac analogs (H6 and H3) were synthesized (FIG. 1). However, the synthesis of Neu5Gc glycans using these methods is still a challenging task. Therefore, an enzymatic method has been extensively used in the synthesis of complex sialylated-glycans.

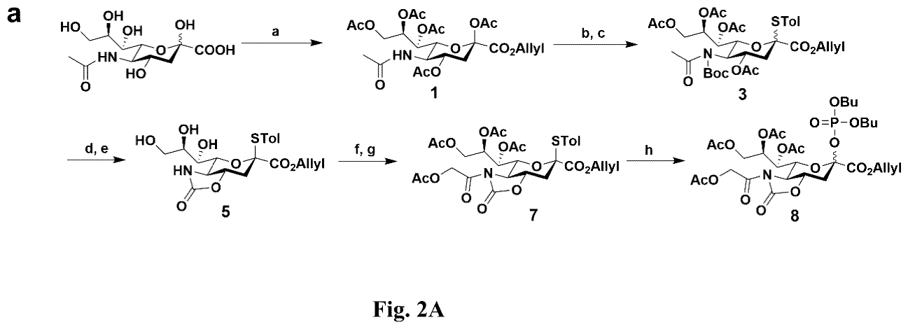

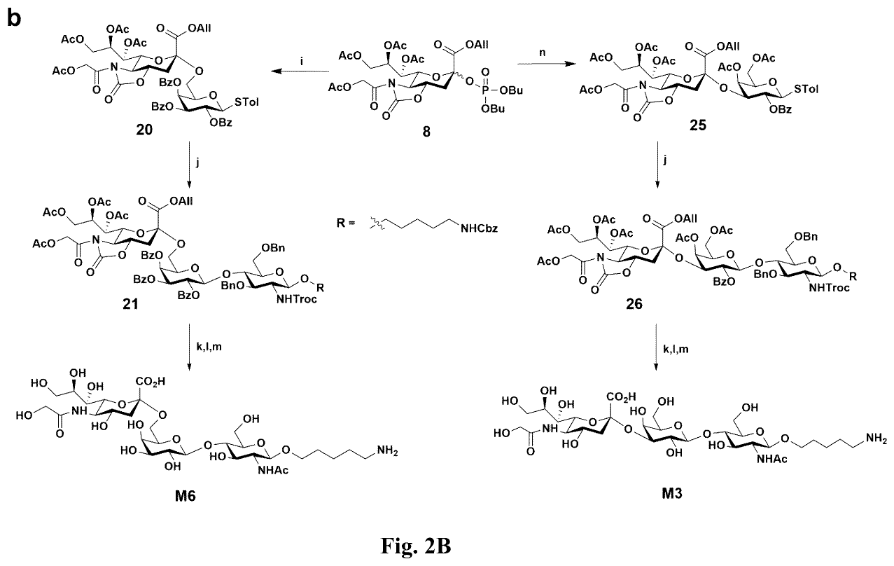

In the synthetic strategy disclosed herein, two key steps were adopted for synthesizing Neu5Gc glycans: (a) sialic acid glycans were developed using allyl ester instead of traditional methyl ester-ligand to avoid harsh deprotection conditions, which may cleave α-sialyl linkage; (b) a labile method was developed to deprotect oxazolidinone ring to control the selective N-glycolyl substitution. Neu5Gc glycans were obtained from orthogonally protected sialic acid donor 8 and sequentially glycosylated to galactose and glucose building blocks under standard glycosylation conditions FIG. 2. The sialic acid donors were obtained by single step peracetylation and allyl-esterification of sialic acid, followed by p-thiocresol glycosylation and Boc-protection. Deacetylation of 3 in the presence of sodium methoxide and allyl alcohol, followed by oxazolidinone formation and peracetylation yielded desire donor 5, which was treated with acetoxyacetyl chloride to obtain Neu5Gc 7 thio-donor. Finally, glycosylation of 7 with dibutyl phosphate in the presence of N-iodosuccinimide (NIS) and trifluoromethanesulfonic acid (TfOH) yielded the desire sialic acid donor 8 in an excellent yield (FIG. 2).

To achieve a (2-6) and a (2-3) glycosylated sialic acid disaccharides 20 and 25, two different galactose building blocks 12 and 17 were synthesized in 5 and 8 steps from D-galactose (ESI). The glucose building block 18 was synthesized and glycosylated with Cbz-amine protected linker using standard glycosylation conditions to obtain 82% of 19 (ESI). The sialic acid disaccharides (20, 25) was obtained by glycosylating the sialic acid donor 8 with 12 and 17 acceptors in the presence of TMSOTf at −50° C. in DCM solvent (FIG. 2B). In the case of α-(2-3) disaccharides, the glycosylated product was again reacted with acetic anhydride to block 4-OH group on galactose residue. Then glycosylation of disaccharide thio-donors (20 and 25) with 19 acceptor was carried out with NIS/TfOH at −20° C. gave protected trisaccharide in moderate to good yield (FIG. 2B). To accomplish the final deprotected M6 and M3, the correct order of deprotection is critical to obtain Neu5Gc analogs. It was found that the oxazolidinone deprotection prior Troc-removal resulted in partial deprotection of Troc. In addition, the global deprotection of oxazolidinone, acetate and benzoyl group using strong basic conditions also resulted complete deprotection of glucose N-acetate. Thus, Troc-protection removal and acetylation are the first necessary step to maintain the N-glycolyl group. This was followed by selective oxazolidinone deprotection using 1,2-ethanethiol and DBU mixture, followed by global deprotection using lithium hydroxide, followed by hydrogenolysis yielded (M6) and (M3). The trisaccharides (H6, H3, M6, M3) were synthesized with a primary amine at the terminus of the extending linker (FIG. 1). This enables electrochemical grafting on glassy carbon electrode (GCE) or amidation with lipoic acid on modified Au electrode (AuE).

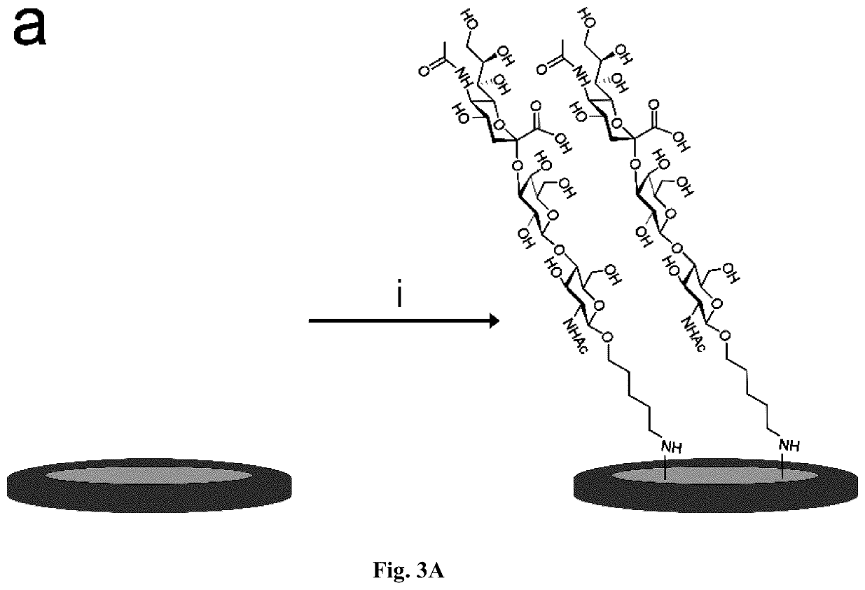

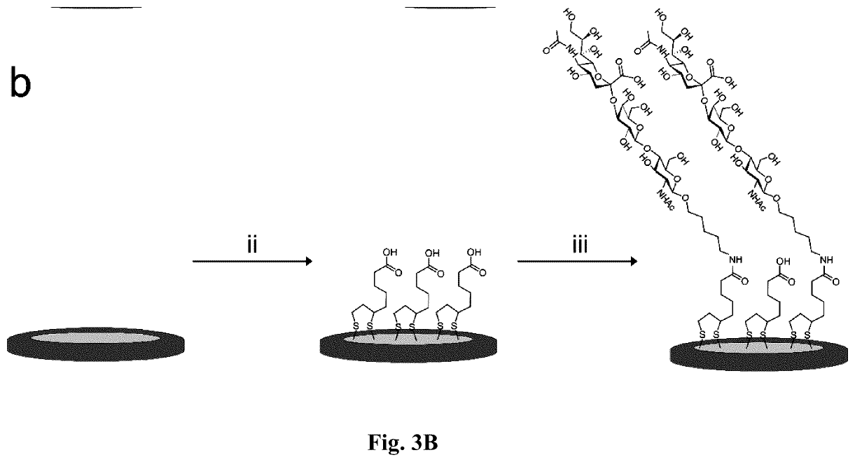

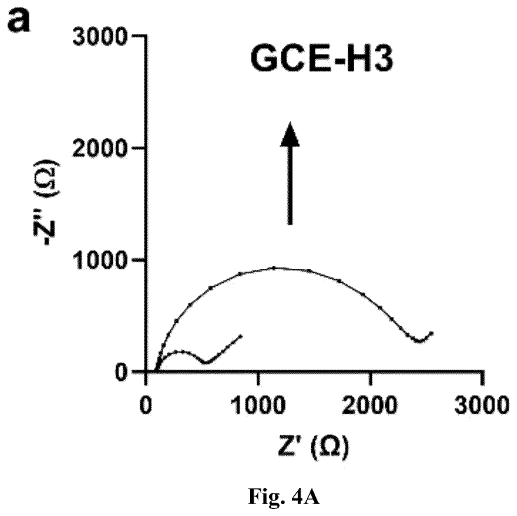

The substrates H6, H3, M6, and M3 were electrochemically grafted with the GCE by applying 5 cycles of CV with a scan rate of 0.01 V/s from 0.6 to 1.2 V referenced to Ag/AgCl (3M KCl) electrode to give GCE-H6, GCE-H3, GCE-M6, and GCE-M3 respectively (FIG. 3A). This resulted in charge transfer resistance (R_{CT}) increase to an approximately value of 50002 after the deposition suggests grafting with the glycan. To support this claim, deposition with the same condition was performed on glassy carbon plates (GCP) with H6 to give GCP-H6. These plates were characterized by CPD and CA analyses. A decrease in CA from 75° to 55° suggests the addition of hydrophilic molecules on the surface. The increase in V_{CPD} from −315 to −106 mV (ΔCPD=+209 mV) suggests the addition of negative charges, which are correlated with the deprotonated carboxylates of the sialic acid. The collective data suggest that the saccharides were electrografted on the GCE. Therefore, they can be further evaluated for impedimetric analyses.

Another system was prepared by modifying AuE to give broader insight on the enzymatic reaction in an interface. AuE were modified with lipoic acid chemisorption and amidation with H3, H6, M3, and M6. Each step of modification showed an increase in the R_{CT} suggesting modification of the AuE. VASE analyses of the modification and Au surfaces showed the formation of a layer with a thickness of 5 Å, CA of 60°, and V_{CPD} of −192 mV following chemisorption of LPA. Coupling of the trisaccharide resulted in an increase of the layer thickness to 13 Å and decrease of V_{CPD} to −232 mV (ΔCPD=+40 mV) suggesting the formation of a sensory layer with the saccharide on AuE. However, the hydrophobicity of the layer remained unchanged with CA of 60°.

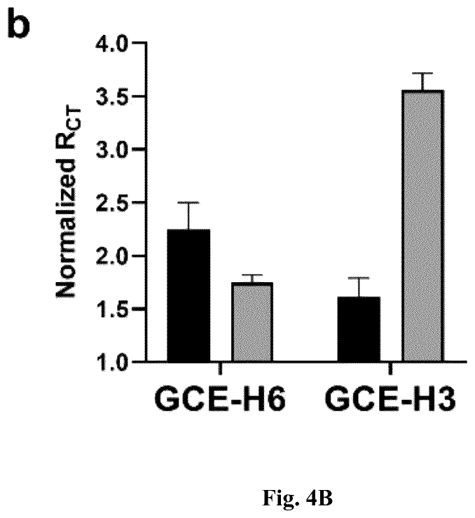

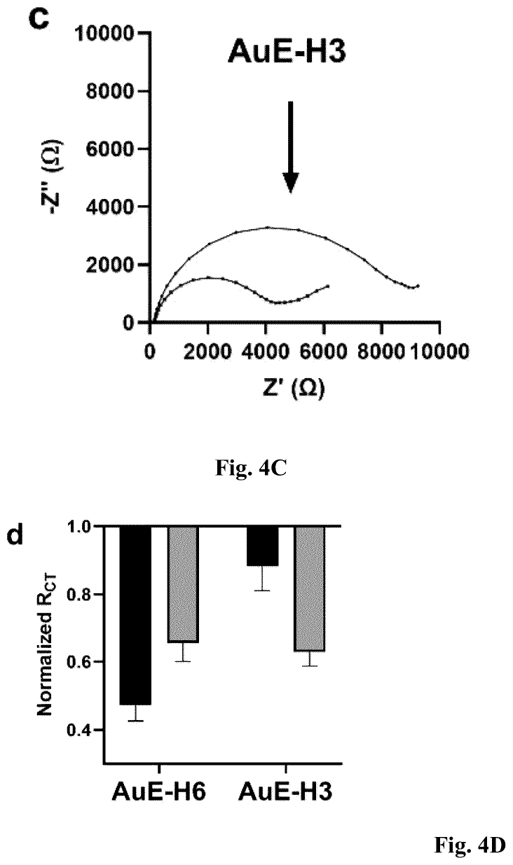

EIS analyses were performed on GCE-H6 and GCE-H3 prior and after exposure to 3 mU/mL of two types of NA and the R_{CT} was normalized to the initial value obtained from the Nyquist plot (FIGS. 4A and B). The first NA, which has preference for 2,3 bond cleavage, is isolated from Clostridium perfringens (3NACP) and the second NA, which prefers 2,6 bond cleavage, is isolated from Arthrobacter ureafaciens (6NAAU). GCE-H6 showed a preferential response to 6NAAU while GCE-H3 showed a preferential response to 3NACP. These results are in line with the reported enzyme sialosides specificity.

EIS analyses were performed on AuE-H6 and AuE-H3 prior to and after exposure to 3 mU/mL of the two types of NA (FIG. 4C). In the case of the modified AuE, a decrease in the R_{CT} value was observed with the same substrates attached to GCE. The response was summarized in FIG. 4D. The relative responses of AuE-H6 and AuE-H3 were preferential to 6NAAU and 3NACP respectively. This shows that there is a preference of electrode bound substrate to the proper enzyme independently of the surface used and the signal behavior and that EIS may be used to evaluate electrochemically enzyme preference and distinguish between NA.

Surface characterization techniques were used to evaluate the source of the impedimetric signal resulting from exposure of enzyme to substrate on GC and Au. XPS, CPD, and CA analyses were performed on GCP-H3 before and after exposure to 3NACP. A relative increase in N1s peak at 400.4 eV related to amides and C1s at 288.9 eV related to carbonyls suggests that the enzyme is adsorbed on the surface of the modified GCP. Change in CPD by −198 mV to a value of −304 mV after exposure to 3NACP, which is very close to the initial value of −315 mV, implies that there is an addition of a dipole that cancels the previous effect. This non-oriented dipole can be related to the addition of the enzyme. An increase of CA to 82° also suggests a change in the interfacial properties. Assuming that there is an addition of the enzyme on the surface, the increase in hydrophobicity can be related to the adsorption of the protein with hydrophobic functional groups pointing towards the interface. VASE analyses of exposure of Au-H3 to 3NACP showed no change to the layer thickness, which remains 13 Å thick, and a small increase in hydrophobicity to 66° and V_{CPD} to −215 mV. Additionally, XPS showed no addition of amide bonds related to the enzyme backbone after exposure. These analyses indicate that the enzyme is not adsorbed to the gold layer, hence, the observed response can be related to enzymatic reaction. The surface characterization analyses imply that the observed impedimetric response on GCE is related to specific adsorption of the enzyme whereas the response on AuE is related to the enzymatic reaction.

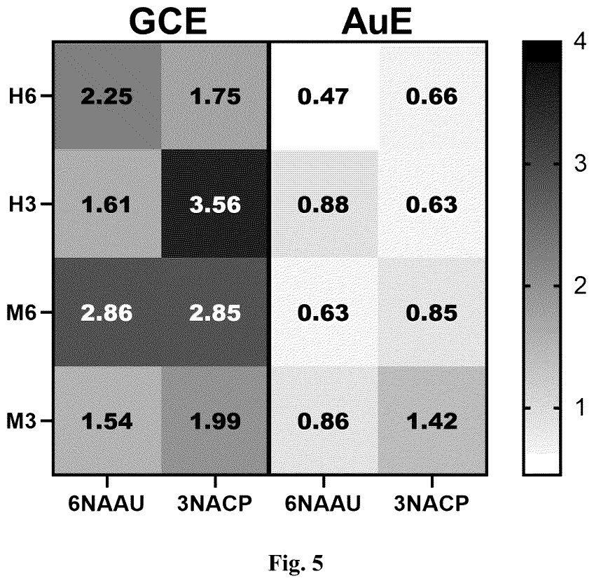

To further explicate the enzyme binding and reaction preferences on sialosides that derive from different animal, GCE-M6, GCE-M3, AuE-M6, and AuE-M3 were exposed to 6NAAU and to 3NACP and impedimetric analyses were performed. While GCE-M3 showed slight preference to 3NACP, GCE-M6 did not show preferential response to any of the NA (FIG. 5). This suggests that the binding preferential response is not only related to the position of the SA but also to its type or origin. Impedimetric control experiment was carried out by exposing 3NACP to GCE modified by same procedure with propyl amine. The exposure resulted in absence of significant change in R_{CT}, hence, indicating that the glycan is required for the recognition event. The enzymatic response using AuE-M6 showed a clear preference to 6NAAU as opposed to GCE-M6 (FIG. 5). This can be related to the interfacial interactions combined with interaction with the substrate that cause differentiation between the enzymes only on AuE. AuE-M3 showed low response to the unfavored enzyme 6NAAU that is in line with enzymatic preference. However, AuE-M3 showed an increase impedance when exposed to 3NACP, which is opposite signal trend to other AuE-based systems. This may be attributed to specific adsorption of 3NACP on AuE-M3 and not to enzymatic reaction. XPS analyses showed that there is a slight increase in the amide signals on AuE-M3 from adsorption of enzyme. This explains the observed increase in impedimetric response in the case of AuE-M3 response to 3NACP.

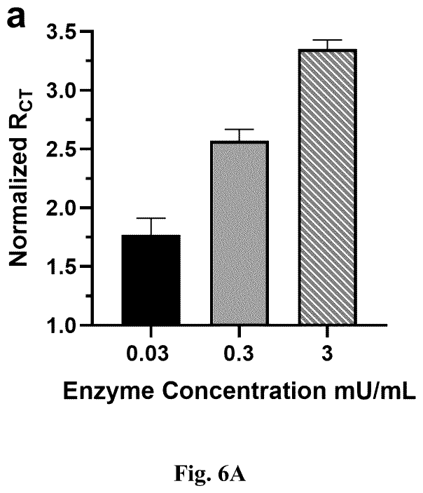

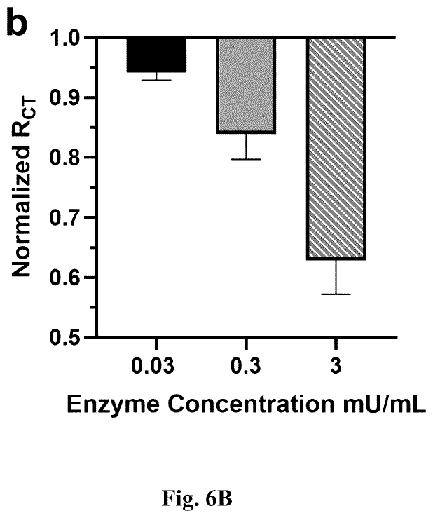

To evaluate the sensing sensitivity of the platform, GCE-H3 and AuE-H3 were exposed to 3, 0.3, and 0.03 mU/mL of 3NACP. The normalized response with different concentration of the NA was plotted (FIG. 6). These results show that there is a concentration-dependent behavior for the adsorption on GCE and the enzymatic reaction on AuE. The enzymatic reaction dependent system was insensitive to low concentrations of enzyme as opposed to GCE, hence, GCE based system has higher sensitivity for enzyme detection.

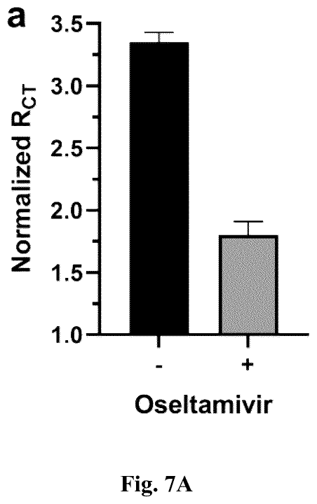

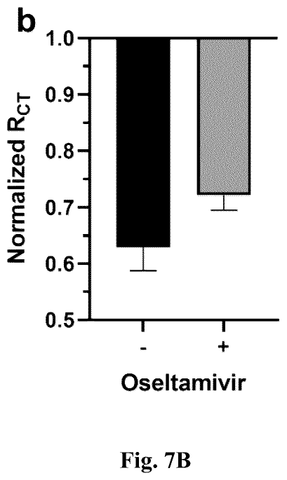

To evaluate the effect of NA inhibitor on the affinity to the modified GCE, GCE-H3 was used. GCE-H3 was exposed to 0.3 mU/mL 3NACP in the presence of 0, 0.1, and 1 μM of the antiviral drug Oseltamivir (FIG. 7A), which is a known NA inhibitor. Addition of 0.1 μM of Oseltamivir resulted in lower adsorption response of GCE-H3 to 3NACP. Increase in Oseltamivir concentration to 1 μM resulted in further decrease in response to the enzyme. The adsorption inhibition following exposure to Oseltamivir suggests that the competition on the catalytic site cause the decreased affinity to the sensory layer with the glycan. Combining these results with the deferential affinity to the proper substrate suggests that the enzyme major interaction is with substrate. There is a probability for additional interaction with the GCE that prevents the enzyme detachment from the interface as was observed by surface characterizations. Additionally, Evaluation of inhibition was performed on AuE-H3 with 3 mU/mL of 3NACP in presence and absence of 1 μM oseltamivir (FIG. 7B). The concentrations that were used on AuE-H3 were higher because there is lower sensitivity in the system when low concentrations are used. The decrease in response using the system of AuE-H3 in the presence of the inhibitor suggests that the enzyme inhibition activity can be sensed and studied utilizing the presented platforms.

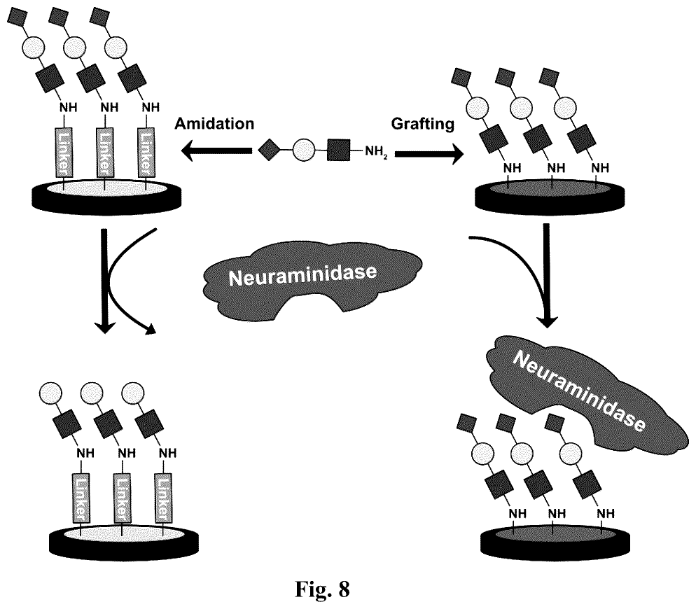

In this study, two platforms based on type of surface were developed for modification with four sialosides. The two platforms bearing the same substrate showed opposite impedimetric response when exposed to the same enzyme. Surface characterization showed that the type of behavior after exposure depends on the modified surface. This is in line with previous work that addressed the effect of surface characteristics on the phenomena occurring in the interfaces. In this work the modified AuE was sensitive to the enzymatic process while the modified GCE was selectively responsive to the presence of the enzyme binding on the surface (FIG. 8). The intensity of the response was correlated with the sialoside preference by enzyme regardless to the platform used. It was observed that the modified GCE has higher sensitivity compared to the modified AuE counterpart. Inhibition analyses showed that both platforms can detect inhibition in binding and reaction, however, the higher sensitivity of binding analyses suggest that GCE is the preferable method for design of NA inhibitors. Both systems are viable for sensing where AuE can be used for detection of the desialylation enzymatic reaction while GCE can be used for a more sensitive detection of NA presence.

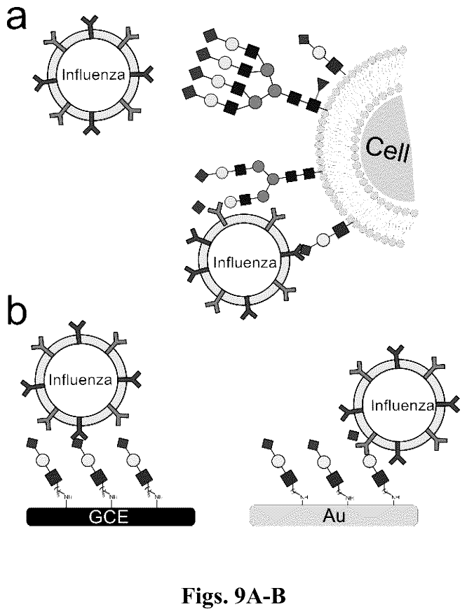

The system of the invention was utilized for impedimetric detection of various types of influenza NA based on the phenomena of binding and catalytic activity (FIG. 9). The results show electrochemically that the design system is sensitive to H1N1 and H3N2 NA, and that it can differentiate between the NA and distinguish them from bacterial NA by analyzing the response of the NA to 4 types of sialoside trisaccharide on two types of interfaces.

### Results and Discussion

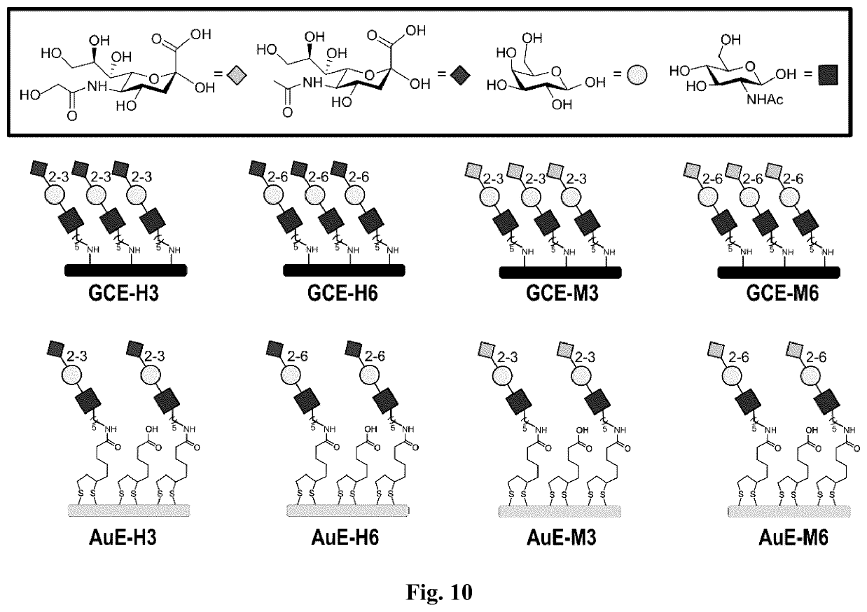

Two platforms modified with synthetic sialoside trisaccharides were developed for the detection of neuraminidase based on binding and enzymatic activity of bacterial NA. The first interface is produced by electro-grafting glassy carbon electrodes (GCE) with Trisaccharide with amine linker at the reducing end to give GCE-H3, GCE-H6, GCE-M3, and GCE-M6 (FIG. 10). The second interface is produced by coupling the amine terminated trisaccharides to Lipoic acid (LPA) monolayer on Au electrode (AuE) to give AuE-H3, AuE-H6, AuE-M3, and AuE-M6 (FIG. 10). The eight modified interfaces showed ability to differentiate impedimetrically between bacterial NA by the variation of response produced in each electrode type, therefore, the have potential in differentiation between viral NA based on affinity and catalytic activity.

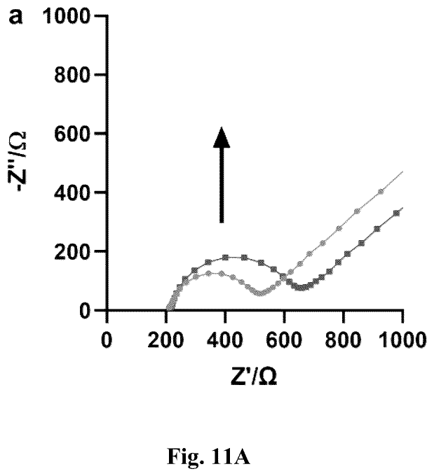

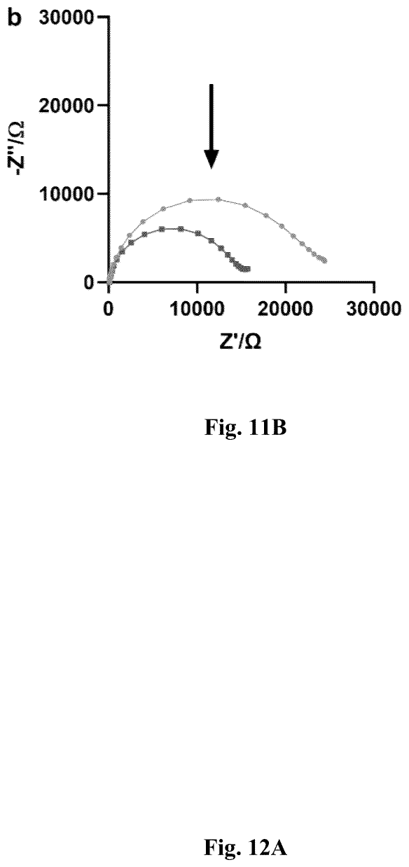

To determine if the systems respond impedimetrically to viral NA in similar manner as to bacterial NA, EIS measurement was performed using GCE-H3 before and after exposure to H1N1 NA. The exposure to the enzyme resulted in increase of charge transfer resistance (R_{CT}) (FIG. 11A), which is in line with our previous observation for NA binding. EIS measurements were also performed using AuE-H3 before and after exposure to H1N1 NA. This exposure resulted in a decrease of R_{CT}, which is in line with our previous observation of NA enzymatic activity on AuE modified with sialosides. To confirm the observation of viral NA binding and activity, XPS analyses were performed on modified Au and GC.

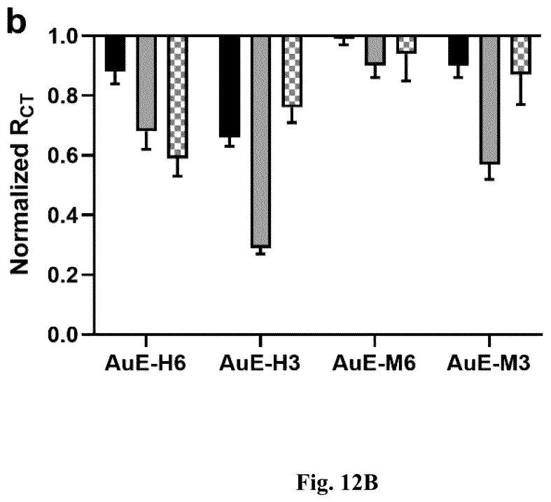

For comparative study of viral NA response by the various platforms, each system was exposed to H1N1 and H3N2 NA and the impedimetric response was recorded, Normalized, and plotted (FIG. 12). Modified GCE responded in increase of R_{CT} in except in the case of GCE-H3 and GCE-M3 with H3N2 NA (FIG. 12A). Unlike the case of H3N2, GCE-H3 and GCE-M3 responded to H1N1 NA. This indicates that these two platforms can detect H1N1 NA but cannot detect H2N3. GCE-H6 and GCE-M6 responded to both H1N1 and H3N2 with similar intensity, indicating that the two platforms can detect both viral NA but cannot differentiate between them. Combination of the responses can be used to both detect the viruses and differentiate between them. Modified AuE were exposed to the two enzymes and the normalized R_{CT} was recorded (FIG. 12B). AuE-M6 showed no response to H1N1 while responded to H3N2. This indicates the AuE-M6 can be used to detect H3N2 but not H1N1. AuE-H3, AuE-H6, and AuE-M3 responded to both H1N1 and H3N2. In all these cases the enzymatic activity sensing was higher for H3N2 indicating faster enzymatic activity in the presented interface. Both H3N2 and H1N1 NA showed the highest activity on AuE-H3, which is in line with previous works on influenza NA substrate preference. This indicates that the presented system can be used also to study viral NA enzymatic activity and not only differentiate between the NA for sensing applications.

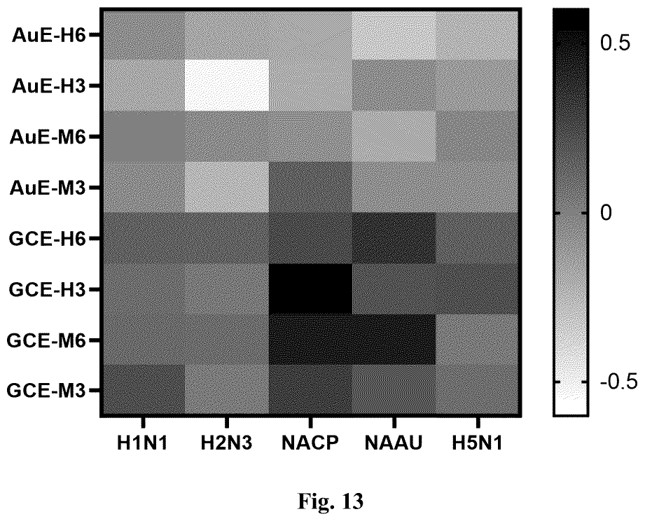

To compare between the responses, Log (normalized R_{CT}) was taken for both influenza NA response in this study and bacterial NA, which are Clostridium perfringens NA (3NACP) and Arthrobacter ureafaciens NA (6NAAU). The calculated results were plotted on a heatmap (FIG. 13). The results show that the bacterial NA has higher response for binding. This can be as result from the difference in NA exposed surface that cause stronger binding by bacterial NA. In the case of activity on AuE the viral NA show higher preferential activity for the correct substrate than bacterial NA. when looking at the structure of viral Neuramindases (PDBid 7S0I and 4H52 for H1N1 and H3N2 respectively), the pocket is located close to the surface of the enzyme with low hydrophobicity surrounding the pocket. This is different from bacterial NA where the pocket is located deeper in the enzyme and the surrounding interface is hydrophobic. The structural differences between the enzyme dictates its affinity to the interface and ability to perform reaction on solid-liquid interface. It is important to note that combination of the eight interfaces for detection of NA showed ability to differentiate between the 4 NA proving to be a powerful tool in infection type detection based on NA activity.

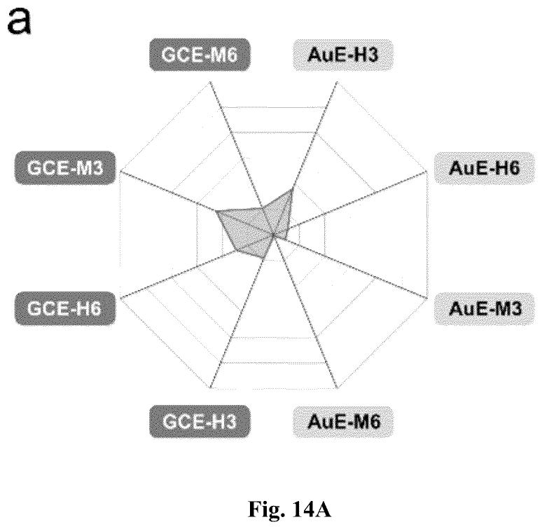

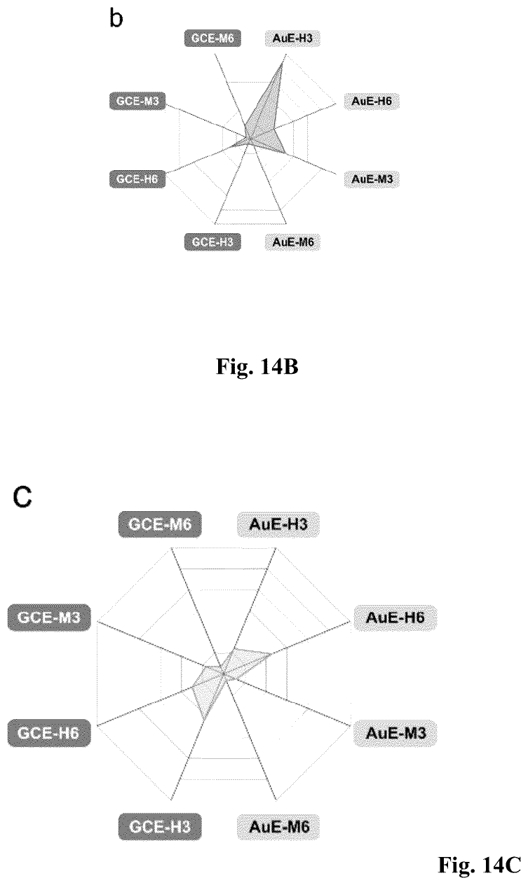

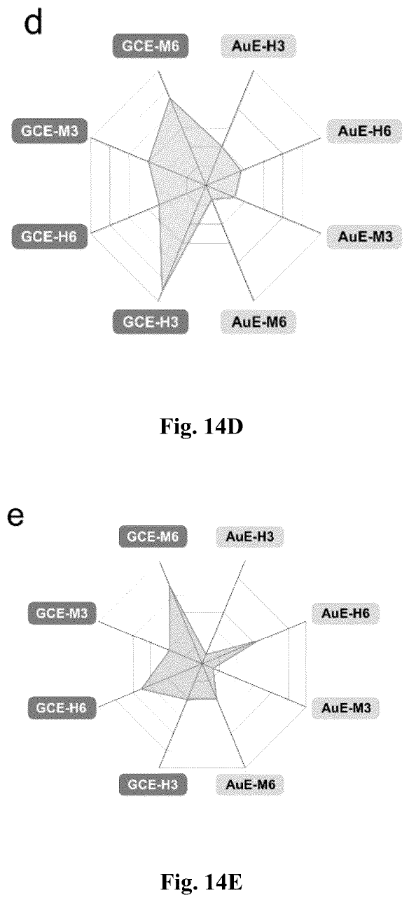

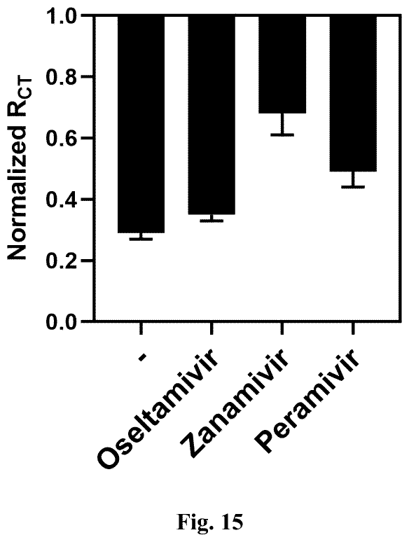

A Radar plot of |Log (Normalized R_{CT})| for the response of H1N1, H3N2, H5N1, NACP, and NAAU with the presented sensory layers is demonstrated in FIGS. 14A-E. FIG. 15 provides the normalized R_{CT} response of AuE-H3 to H3N2 NA in presence and absence of Viral NA inhibitors.

### CONCLUSION

In this part of the work, the inventors have demonstrated the ability to detect and differentiate between viral neuraminidase based on preferential response with sialoside decorated interfaces, which relies on affinity differences to the substrate on an electrode. Results show that binding and catalytic activity of the enzyme was detected by location of the catalytic pocket and surrounding enzyme interface. This causes viral NA, where the pocket is at the enzyme interface, to perform reaction faster on the surface while the bacterial NA, where the pocket is deeper with hydrophobic surrounding interface, to bind stronger the sialosides.

As demonstrated, glassy carbon and Au electrodes were modified by four types of sialylated trisaccharides, which differ by the type of SA and is galactose bound regiochemistry. The modifications were verified by EIS, CPD, and CA analyses, which resulted in changes of R_{CT}, surface potential, and hydrophobicity. The systems were exposed to two NAs with different regiochemistry preferences. This resulted in a preferential signal on the various substrates. Surface characterizations, such as XPS and CPD, support that the changes in impedimetric signals were a result of enzyme adsorption on the surface in the case of GCE and enzymatic reaction in the case of Au. Increase of EIS signal related to the adsorption of the enzyme and decrease for enzymatic reaction proved to be concentration dependent and substrate specific based on glycosidic linkage and type of sialic acid. Additionally, enzyme adsorption and enzymatic reaction in the interface were reduced by the presence of the NA inhibitor Oseltamivir presence. The collective results suggested that the designed platform can be used for rapid detection of NA by an array of small glycan and evaluation of NA inhibitors using both enzymatic sedimentation and enzymatic reaction on the modified interfaces.

### Part II: Discriminating Between NAs

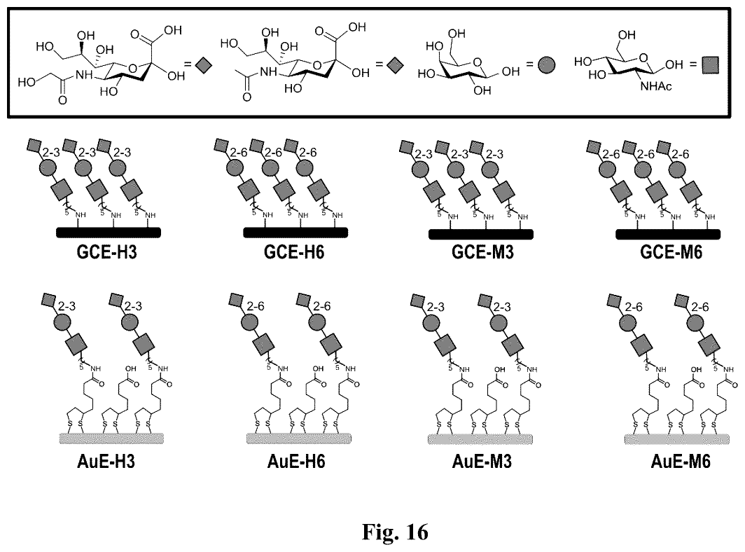

In the above, the inventors demonstrated two platforms modified with a set of four synthetic sialoside trisaccharides for the detection of neuraminidase based on binding and enzymatic activity of bacterial NA. The synthetic trisaccharides differ by the regio-chemistry of sialic acid and by the type of sialic acid and were equipped with an alkylamine linker. The first interface is produced by electro-grafting glassy carbon electrodes (GCE) with the sialosides to give GCE-H3, GCE-H6, GCE-M3, and GCE-M6 (FIG. 16). The second interface is produced by coupling the amine-terminated trisaccharides to Lipoic acid (LPA) monolayer on Au electrode (AuE) to give AuE-H3, AuE-H6, AuE-M3, and AuE-M6 (FIG. 16). The glycated monolayers on Au and GC surfaces differ in several aspects. The AuE-glycated surfaces is negatively charged via the LA sub-monolayer and denser with sialosides compared with GCE. The sialoside diluted GCE-glycated surface is more hydrophobic via the exposed glassy carbon. The eight modified interfaces showed an ability to differentiate impedimetrically between bacterial NA via two distinctive interaction modes. While on AuE the electrochemical signal was the result of catalytic activity on the sialosides the response on GCE results from adhesion of the enzyme to the glycated layer. We aimed to use the same strategy to discriminate between IV NAs from different strains and compare them to the bacterial ones.

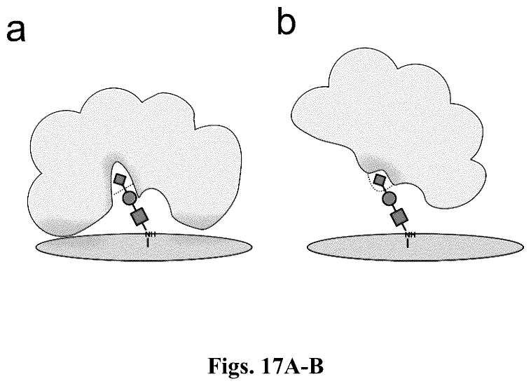

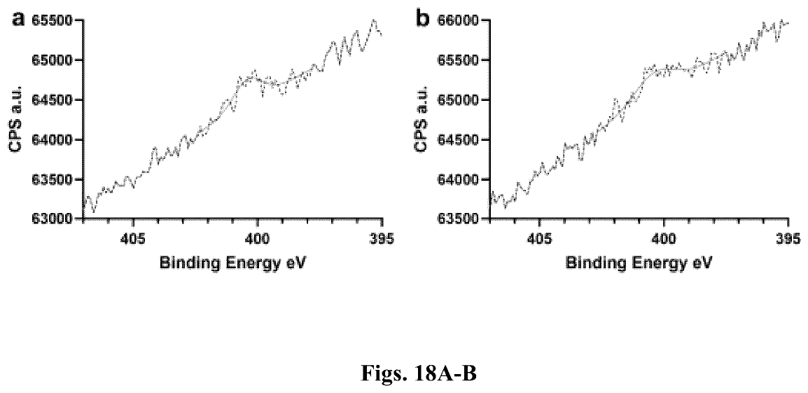

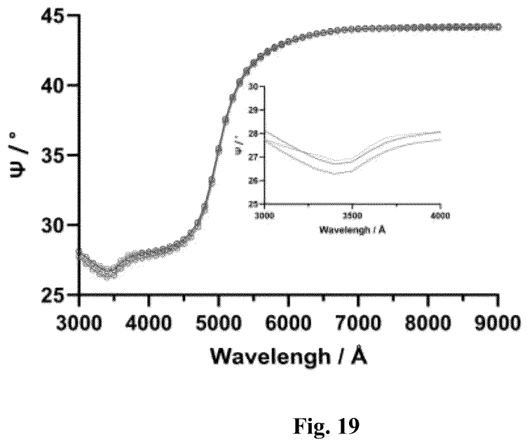

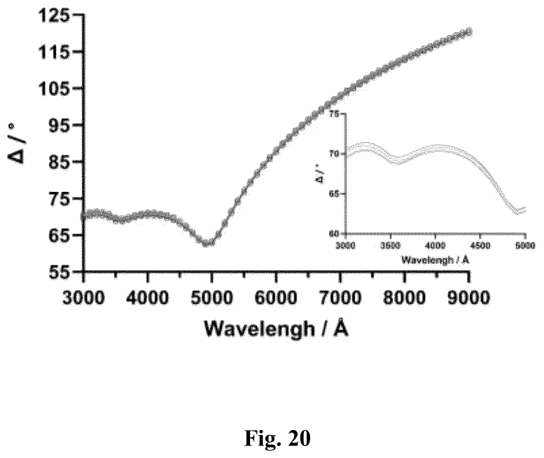

To determine if the systems respond impedimetrically to IV NA, in a similar manner as to bacterial NA, EIS measurement was performed using GCE-H3 and AuE-H3 before and after exposure to H1N1 NA. The exposure of GCE-H3 to the enzyme resulted in an increase of charge transfer resistance (R_{CT}) (FIG. 17A), which is in line with our previous observation for NA binding. The exposure of AuE-H3 to the enzyme resulted in a decrease of R_{CT}, which is in line with our previous observation of NA enzymatic activity (FIG. 17B). To characterize the interaction of IV NA with glycated gold surfaces, X-ray photoelectron spectroscopy (XPS) and variable angle spectroscopic ellipsometry (VASE) analyses were performed prior and after exposure to H3N2 enzyme. The XPS analyses of the nitrogen/amide region after exposure of Au-H3 to H3N2 (FIGS. 18A-B) showed no additional signal of N1s (B.E. 400.1 eV) related to protein amide. VASE analyses showed an increase in the optical thickness to 12 Å from 7 Å following the glycation of the LPA monolayer. No significant change in the optical thickness was observed following the exposure to H3N2 (FIG. 19 and FIG. 20).

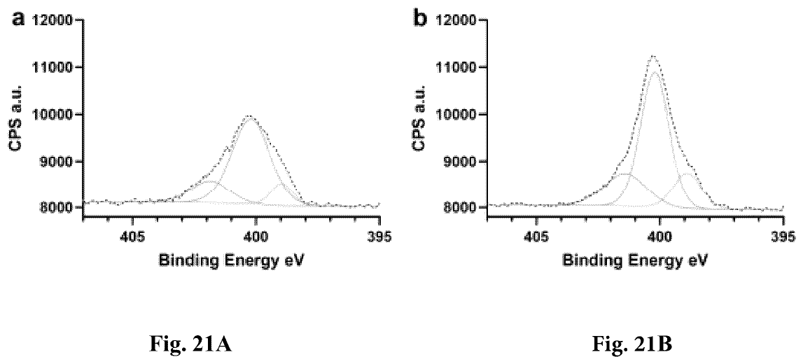

The collective results show that there is no addition of viral enzyme to the gold surface. This is in accordance with the decrease in the electrochemical impedance which correlates with enzymatic reaction. In the case of GCE, XPS analyses performed prior and after the exposure to the enzyme showed the addition of the protein (FIG. 21). However, the increase in the N1s (B.E. 400.1 eV) signal of viral NA is significantly lower compared to bacterial NA.23 This indicates that IV NA has a lower affinity to the glycated GCE surface which is in-line with a small increase in R_{CT}.

To profile IV enzymes from different strains the eight glycated electrodes were exposed to H1N1, H3N2, and H5N1 NAs. The impedimetric response was recorded and the normalized R_{CT} was used to allow a comparison of the different systems. All modified GCE showed an increase of R_{CT} towards the three enzymes. The level of electrochemical impedance increase is both enzyme and substrate-dependent. H1N1 showed a preference to GCE-M3, H5N1 showed a preference to GCE-H3, and H3N2 showed a slight preference for GCE-H6. It was clear that the GCE system can be selective for H1N1 and H5N1 and not for H3N2. This can result from stronger binding responses H1N1 and H5N1 exhibit to glycated GCE.

All modified AuE responded in a decrease of R_{CT} towards the three enzymes. The level of electrochemical impedance decreases again proving both enzyme and substrate dependent. H1N1 showed a preference for Au-H3, H5N1 for Au-H6, and H3N2 for Au-H3. It was clear that the Au system can be selective only for H3N2. Both H3N2 and H1N1 NA showed the highest activity on AuE-H3 compared with the other electrodes, which is in line with previous works on influenza NA sialoside preference. It is noteworthy indicating that for H1N1 and H5N1 show high binding to sialosides with a 2-3 linkage and low catalytic activity. While for H3N2 catalytic activity towards a 2-3 linkages was significant compared to the negligible binding. On the other hand, the a 2-6 linkages didn't show any preference, either catalysis or binding.

While several trends could be established, it was clear that discriminating between the NAs couldn't be done using a single electrode-sialoside pair. The combined effect of each enzyme over a set of glycated electrodes can only be used to differentiate between the NAs. Plotting the various responses on a radar plot where all the analytes respond to all detecting platforms albeit, with different intensities can give another dimension of analysis. Visualization of multiple data points can help to distinguish between analytes with similar biological roles e.g. NAs from different IV strains. To assess the ability to discriminate between the three IV NAs via multicomponent analysis, the |Log (Normalized R_{CT})| response of the EIS towards the entire set of electrodes was combined and presented in radar plots.

Although it is clear that there are overlaps in the response of several electrode-sialoside pairs, the combination of the eight pairs gives a distinctive pattern to each enzyme. It is logical to assume that the affinity and catalytic activity of each enzyme towards different sialosides can never be identical. The radar plot analysis takes advantage of the different R_{CT} signal intensities to provide a powerful identification tool.

In PART I above, the response of the eight electrodes to bacterial NAs, Clostridium perfringens NA (3NACP) and Arthrobacter ureafaciens NA (6NAAU), was studied. To evaluate the characteristic features to distinguish between IV and bacterial NAs, the data is presented in a radar graphs. The comparison shows that the bacterial NA has a higher response for binding compared to enzymatic activity. For IV NA such preference is not as clear and an even stronger response toward catalysis can be observed in some cases. There are structural differences between IV and bacterial enzymes. For bacteria NA, the catalytic pocket is located deeper in the enzyme and the surrounding hydrophobic interface is exposed to the GCE surface which can enhance the affinity to the electrode. The structure of IV NA (PDBid 7S0I and 4H52 for H1N1 and H3N2 respectively), the catalytic pocket is located close to the surface of the enzyme with low peripheral hydrophobicity which in turn does not interact with the GCE surface). On the other hand, the higher catalytic activity on AuE is combined with the fast detachment of the NA from the glycated Au surface and prevents binding. The above observations are in line with the XPS analyses that indicate that bacterial NA binds to hydrophobic sialylated GCE stronger than the IV ones. The EIS on Au shows a catalytic response of the viral NA which is supported by the VASE analysis.

The combination of the responses as manifested in the radar plot provides NAs selectivity. Thus, providing an electrochemical analytical tool to characterize NA of various pathogens. This was achieved by using two types of interfaces enabling us to double the comparison possibilities using scarcely accessible synthetic saccharides. As opposed to microarrays where only one type of interface is accessible for each scarcely accessible. Thus, that shows the advantage of interface modification in selective biosensing.

Impedimetric studies enabled us to assess the decreased bacterial NA catalytic activity on AuE in the presence of NA inhibitor (Oseltamivir). Here, we wanted to evaluate the effect of several NA inhibitors on the EIS response to viral NA. The enzymatic response of AuE-H3 to H3N2 NA was evaluated in the presence of three known inhibitors, Oseltamivir, Peramivir and Zanamivir. The results indicate that Oseltamivir had a weak inhibition effect, Peramivir had a moderate inhibition effect, and Zanamivir had the strongest effect. Clinical trials for influenza A show similar trends in inhibition of influenza by those NA inhibitors. Additionally, the physiological conditions of the patient might influence the efficiency of the anti-viral drug hence the treatment can start with the inhibitor that shows the highest inhibitory activity. Screening methodologies that utilize PCA often rely on large databases. The above results show that a combination of a bio-relevant interaction, e.g. sialoside structure, with an orthogonal surface interaction mode provides a way to rationally increase the database which can lead to enhanced selectivity.

Pathogenic neuraminidases bind many types of sialosides, which makes discrimination based on a single glycan analysis impossible. However, binding and catalysis preference towards different sialosides does exist in terms of affinity and hydrolysis kinetics. The ability to characterize each neuraminidase can hence be enabled by profiling their electrochemical response towards a set of sialoside-based electrodes. We designed a system where each electrode provides three parameters that influence the interaction: sialoside type, regiochemistry, and electrode surface. The developed strategy allows discrimination between NAs and the evaluation of inhibitor efficacy. It might be useful both for determining the infection source and for defining the anti-viral treatment against different viral strains. Standard analytical techniques rely only on sialoside structure to profile NAs. We used the unique NA affinity to the surface and sub-monolayer to provide a glycan-mediated protein-electrode interaction. This provided a new multiparametric electrochemical way to identify each NA even by using a limited set of sialosides. The surface properties and their role in biochemical analysis are generally overlooked. However, those exact properties can be used to give an additional dimension to these interactions. Considering the complexity of glycan synthesis, providing means to enhance the amount of data without increasing the synthetic load is extremely important. We demonstrated here that the surface interaction adds useful data crucial for characterizing protein families that target similar moieties. This new paradigm in array biosensing suggests that, in the future, assembling the same set of receptors on a variety of surfaces will enhance and improve the bioinformatics data.

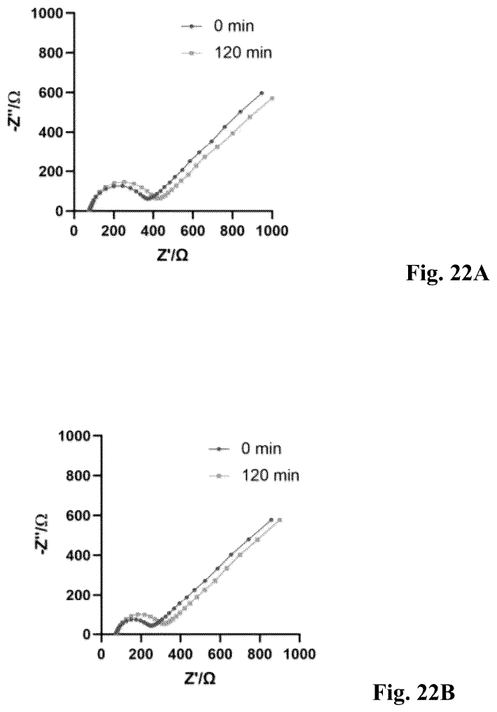

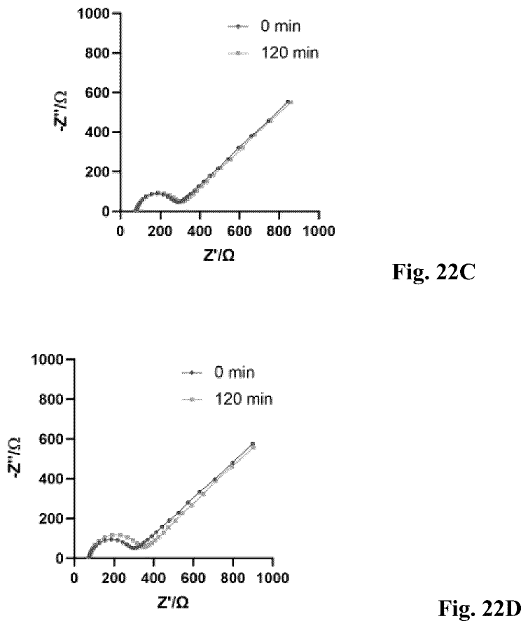

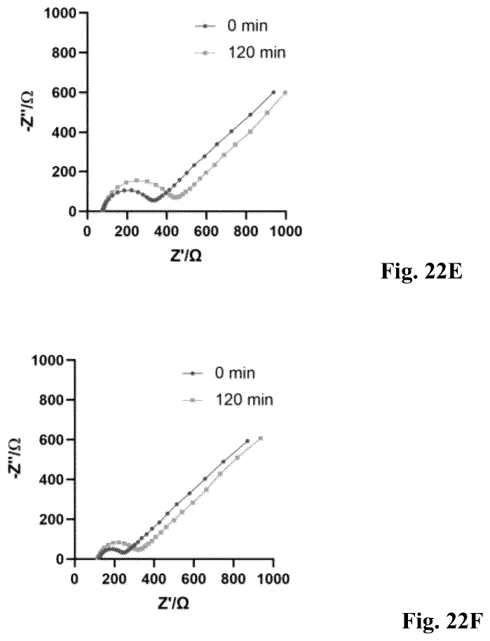

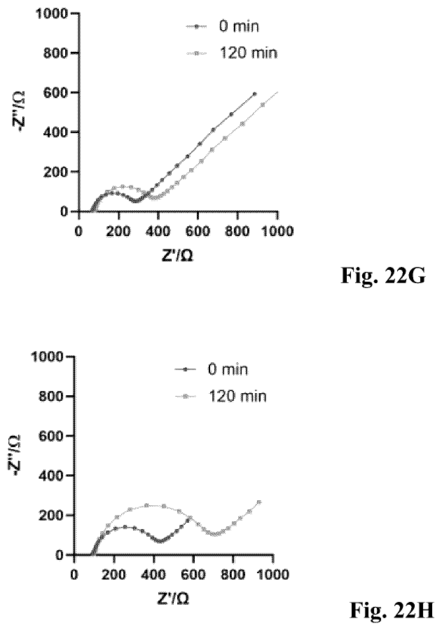

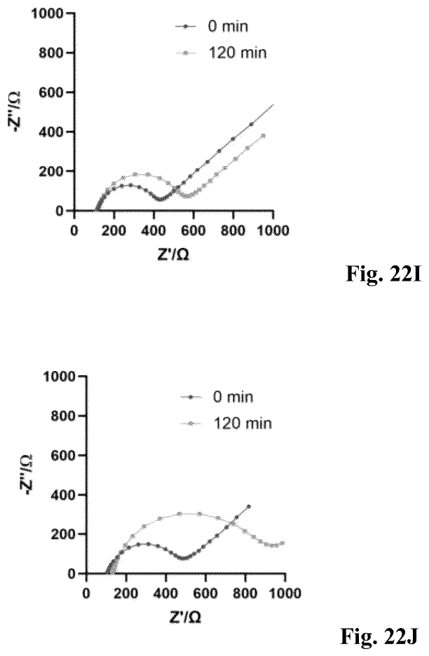

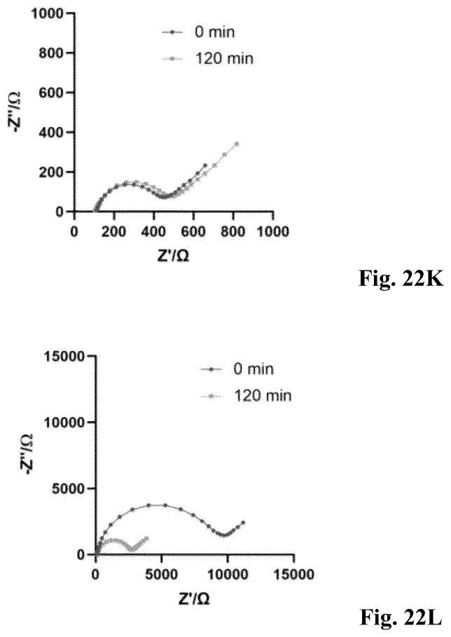

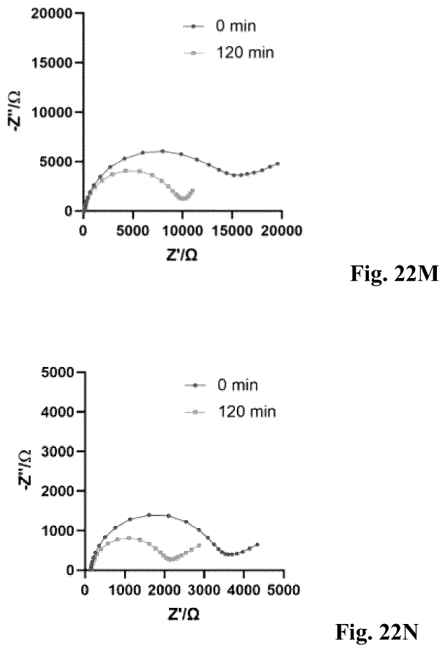

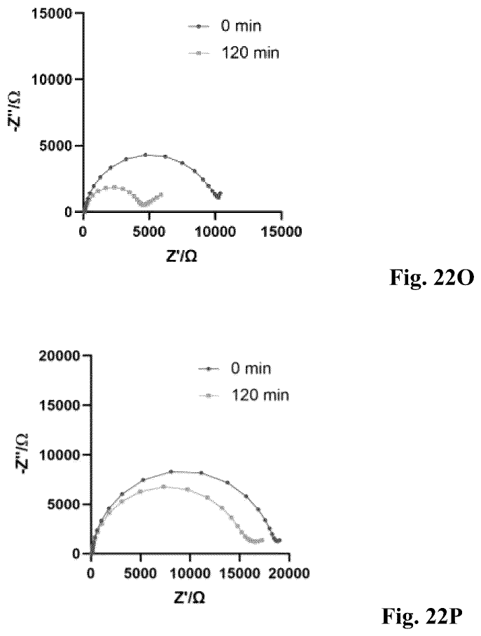

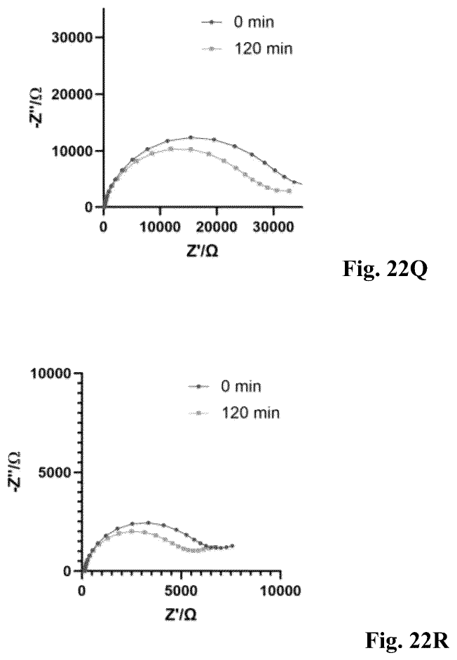

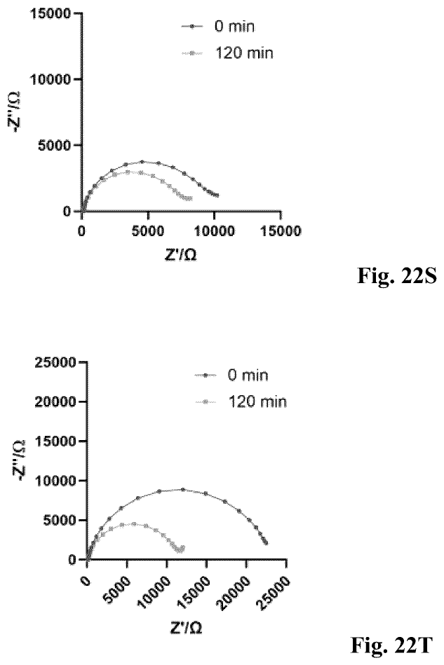

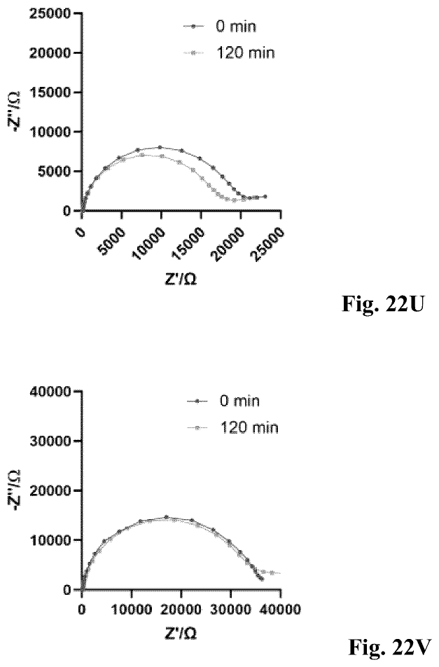

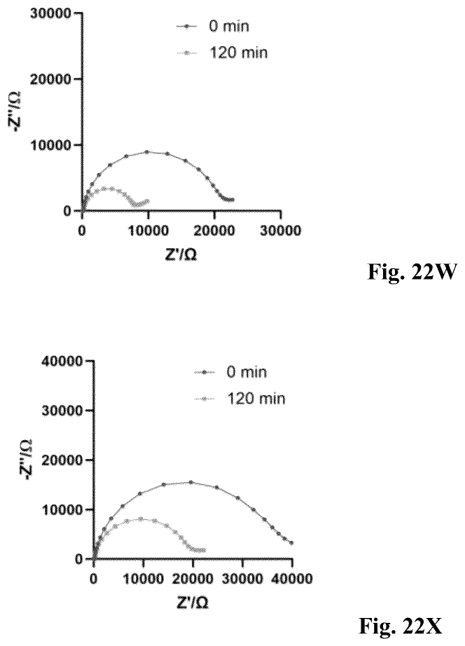

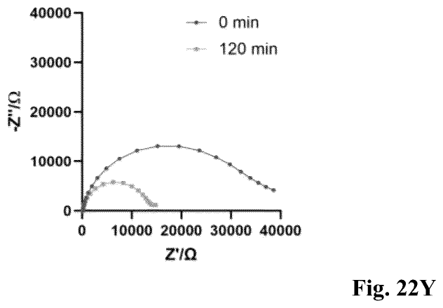

FIG. 22 represents raw data of the spider plots discussed herein. These curves include the capacitance and admittance parameters which can provide additional “spiders” diagrams, paving the way for further dimensions of analysis which using AI tools can enhance selectivity.

1. A sensor array for discriminating between neuraminidases (NA) of different origins using electrochemical impedance spectroscopy (EIS), the array comprising a plurality of different glycated sensors or sensing regions, each different sensor or sensing region being distinguishable in sensor surface material and glycated material.

2. The sensor array according to claim 1, being an EIS electrode.

3. The sensor array according to claim 1, wherein the different sensor surface is selected from a gold surface, a glassy carbon surface (GC), indium-tin-oxide (ITO), chromium, graphite, silicone, single wall and multi wall carbon nanotubes (CNT), silver, iron oxide (Fe3O₄), graphene and conducting polymers.

4. The sensor array according to claim 1, wherein the sensor surface is gold or GC.

5. The sensor array according to claim 1, wherein the glycated material is selected from different sialylated glycans differing in a sialic acid origin and connectivity.

6. The sensor array according to claim 4, wherein the glycan is a disaccharide or a trisaccharide, or a higher saccharide comprising one or more Gal and/or Glc units.

7. The sensor array according to claim 5, wherein the glycan comprising β-D-Gal-(1-4)-β-D-GlcNAc-(1-4))-1-X, wherein X is selected from a C2-C10alkyl, C2-C10alkenyl, C2-C10alkynyl, a C6-C10arylene, or a C2-C10alkyl-C6-C10aryl, each being substituted by an atom or a group of atoms, being one or more of halogens (I, Br, Cl or F), hydroxy (OH), thiol (SH), disulfide (—S—S—), cyano (CN), nitro (NO2), azide (N3), amine (NH2, or a primary, secondary or tertiary amine), carboxy (COO), or a phosphate group.

8. The sensor array according to claim 1, wherein the glycated material is a sialoside of a structure selected from:

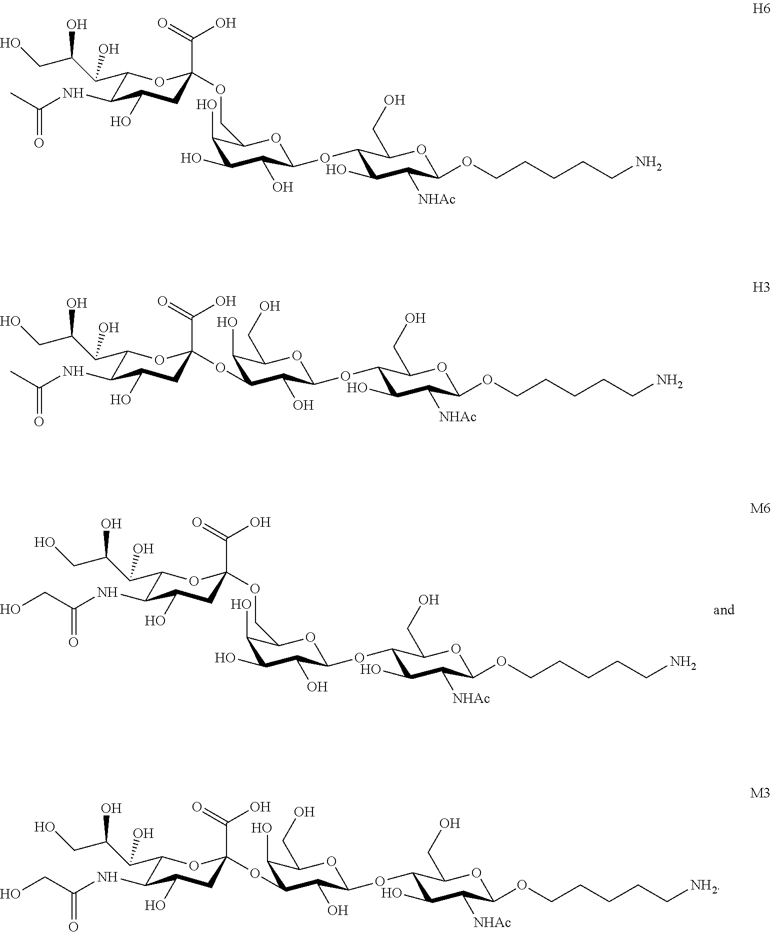

9. The sensor array according to claim 1, comprising a plurality of different sensors or sensing regions, the different sensors having a surface material being a gold surface or a GC surface and a glycated surface comprising a material selected from: 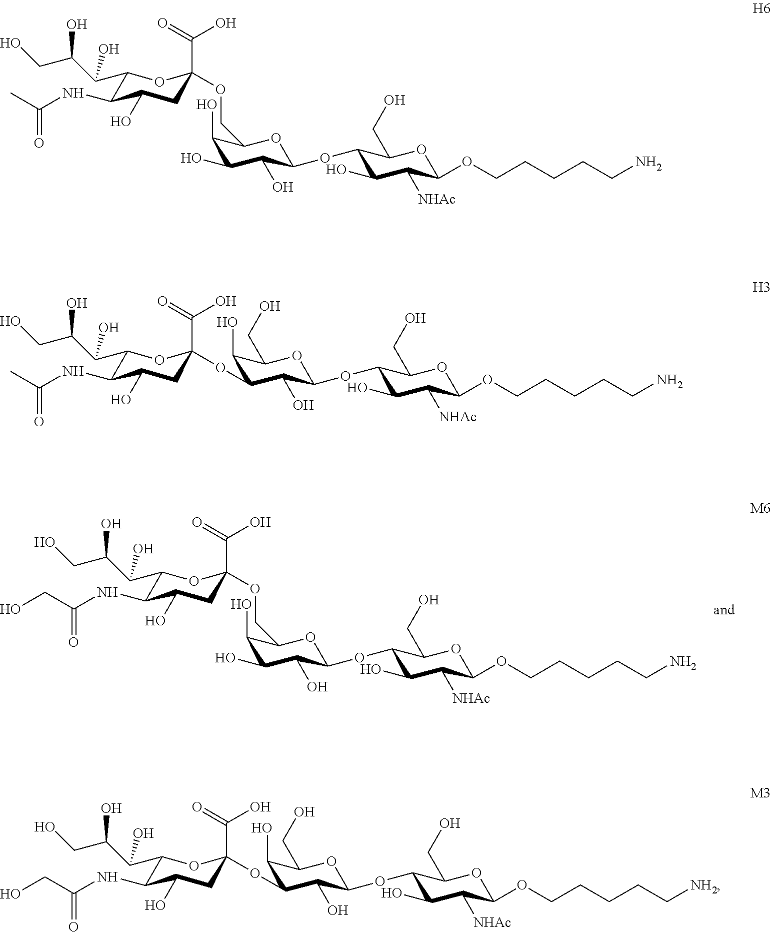
such that the sensor array comprises a plurality of different NA responsive sensors or sensing regions.

10. The sensor array according to claim 1, wherein the plurality of different glycated sensors or sensing regions comprising a plurality of gold sensors and/or a plurality of glassy carbon sensors associated with two or more different glycated material.

11. The sensor array according to claim 1 comprising two or more different sensors or sensing regions.

12. A system for detecting and determining presence or nature of at least one NA, the system comprising a plurality of sensors or sensing regions, each sensor or sensing regions having a predefined glycated surface configured to detect an impedance signal indicative of a respective NA; a library of impedimetric signals, wherein the library comprises a collection of predefined impedance response patterns corresponding to different NAs; a signal processing unit in communication with the sensor array, the processing unit configured to compare the impedance signals from the sensors to the impedance signals in the library to identify the detected NA, wherein the sensor array is adapted to provide a real-time impedance response that is matched to one or more impedance signals from the library, thereby enabling identification of the NA based on the impedance response.

13. A detection device comprising an a sensor array according to claim 1.

14. The device according to claim 13, for measuring a change in an electrochemical impedance of a surface, wherein the sensor array is configured for detection of a change in an interfacial property related to a biorecognition event occurring on the surface of the electrode in view of an interaction of the sialylated electrode surface with a NA.

15. A method of discriminating between different NAs, the method comprising contacting sensor array with a sample comprising one or more NA comprising, the sensor array comprising a plurality of sensors or sensing regions, each sensor or sensing regions having a predefined glycated surface configured to detect an impedance signal indicative of a respective NA; comparing the impedance signal to impedance signals previously identified for the different NAs, to thereby identify an NA present in said sample.

16. The method according to claim 15, wherein the sensor array is a sensor array comprising a plurality of different glycated sensors or sensing regions, each different sensor or sensing region being distinguishable in sensor surface material and glycated material.

17. The method according to claim 15, wherein the sensor array comprises a plurality of different glycated sensors or sensing regions comprising a plurality of gold sensors and/or a plurality of glassy carbon sensors associated with two or more different glycated material.

18. The method according to claim 15, wherein the sensor array comprises different sensor or sensing region distinguishable in sensor surface material and glycated material.
